(19) United States
(12) Patent Application Publication
(10) Pub. No.: US 2026/0193676 A1
(43) Pub. Date: Jul. 9, 2026

(54) Adeno-Associated Virus Production Platform

(71) Applicant: MedImmune, LLC, Gaithersburg, MD (US)

(72) Inventors: Abdou NAGY, Gaithersburg, MD (US); Albert SCHMELZER, Gaithersburg, MD (US); Lina CHAKRABARTI, Gaithersburg, MD (US); Soojin HAN, Gaithersburg, MD (US)

(21) Appl. No.: 19/134,233

(22) PCT Filed: Dec. 1, 2023
(86) PCT No.: PCT/US2023/082009

§ 371 Date: May 30, 2025

Related U.S. Application Data
(60) Provisional application No. 63/385,750, filed on Dec. 1, 2022.

(51) Int. Cl. C12N 15/864; C07K 16/00; C12N 5/071; C12N 5/10; C12N 7/02; C12N 15/869

(52) U.S. Cl. CPC C12N 15/8645; C07K 16/00; C12N 5/0682; C12N 5/0686; C12N 5/10; C12N 7/025; C12N 15/869; C12N 2500/90; C12N 2510/02; C12N 2510/04; C12N 2710/16043; C12N 2710/16044; C12N 2710/16052; C12N 2750/14143; C12N 2750/14152

(57) ABSTRACT

Provided herein is a novel rAAV-based HSV production method that includes two engineered cell lines: engineered CHO cells to produce multiple AAV serotypes and engineered BHK-21 to produce rHSV-1 stocks that are used in the production of rAAVs in CHO cells. The developed method provides a scalable, serum-free manufacturing platform.

### BACKGROUND OF THE DISCLOSURE

Recombinant adeno-associated virus vectors (rAAVs) are the leading platform for gene delivery, with three licensed products currently approved by December 2021 (Bulcha et al., 2021). rAAV have several advantages as gene delivery vectors, including their ability to transduce variety of proliferating and non-proliferating cells, accommodate cell/tissue-specific promoters, and elicit diminished immune response in comparison to other viral vectors (Kang et al., 2009). AAV is a small, nonenveloped virus in the genus Dependovirus within the family Parvoviridae (Srivastava et al., 1983; Daya and Berns, 2008). The AAV 4.6-kb single-stranded DNA genome contains two viral genes: rep and cap. These genes can be removed and replaced with a cassette expressing a therapeutic transgene along with the necessary rep and cap genes provided in trans (Becerra et al., 1988). AAVs capsids are icosahedral and assembled from 60 viral proteins (VP) monomers with around 5 copies of VP1, 5 copies of VP2 and 50 copies of VP3 (Van Vliet et al., Methods Mol. Biol. 2008; 437:51-91.)

Currently, there are different cell culture expression platforms for rAAVs, including stable packaging cell lines, which express the rep and cap genes of a desired rAAV serotype, stable proviral cell lines that stably express Rep, Cap, and the transgene, and triple transient transfection (Clark et al, 1995, Clark., 2002, Qiao et al, 2002). The triple transient transfection is the most common method for rAAVs production, which uses three plasmids: one encoding the gene of interest (GOI) flanked by the AAV inverted terminal repeat (ITR), a second encoding the rep and cap genes of AAV, and the third encoding the adenovirus helper function genes. This three-plasmid system can be simplified to a co-transfection by inclusion of the helper genes in the rep-cap plasmid (Grimm et al., 1998 and Clark K. R., 2002). However, the triple transient transfection method is challenging to scale-up for very high doses, resulting in low specific yields of infectious particles (ip), and often result in high DNase resistance particles (DRP/ip) ratios (50-100) (Grimm et al., Hum Gene Ther; 9: 2745-2760 (1998) and Zolotukhin et al., 2002 Methods; 28:158-167).

Like adenovirus, it has been reported that recombinant HSV-1 (rHSV-1) can also support rAAVs replication as part of a helper virus system (Buller et al, 1981 J Virol; 40: 241-247). The minimum HSV helper genes required for rAAVs replication are the HSV helicase primase complex (UL5, UL8, UL52), the HSV DNA polymerase, and the HSV DNA-binding protein (UL29) genes (Weindler and Heilbronn, 1991 J Virol; 65: 2476-2483). Moreover, it has been reported that the rHSV co-infection method that used two HSV-1 vectors, one encoding the desired rAAV rep/cap serotype and the second encoding the GOI, generated very high specific yields of rAAVs of multiple different serotypes, with a very low (DRP/ip) ratio, in various cell lines (Kang et al, 2009). Another advantage for HSV helper system is that HSV is capable of replicating and providing helper functions in different mammalian cells, which means HSV helper functions bypass the host range restriction for rAAVs production, compared to adenovirus helper-assisted rAAV production that requires human cell lines to succeed (Buller et al., 1970 J Gen Virol; 43: 663-672).

Chinese hamster ovary (CHO) cells are predominantly used as expression hosts for production of recombinant monoclonal antibody (mAb) and other therapeutic proteins, which comprises the fastest growing segment of the biopharmaceutical industry (Walsh G., 2018 Nat. Biotechnol; 36:1136-1145). Due to the regulatory acceptability and low manufacturing cost of CHO cells relative to human cells, using CHO cells for viral production is desirable. However, there is limited experience, if any, in literature about the use of CHO cells for rAAV production, likely due to cellular restriction factors in these cells that may affect rAAV production and interfere with virus packaging. For example, it has been shown that CHO cells did not support vaccinia virus replication at the stage of viral intermediate protein synthesis (Ramsey-Ewing and Moss. 1995 Virology. 1995 Feb. 1; 206(2):984-93). In addition, CHO cells are naturally resistant to HSV-1 productive infection because of the lack of key receptors for HSV-1 entry and infection (Montgomery et al., 1996). Therefore, there is a need in the art to engineer serum free-adapted suspension CHO CAT-S cells to be permissive for infection with incompetent HSV-1 vectors that encode necessary elements for diverse rAAV production.

### SUMMARY OF THE DISCLOSURE

The present disclosure is directed to a baby hamster kidney (BHK) cell that is adapted to grow in serum-free conditions, wherein the cell stably expresses a hamster codon-optimized herpes simplex virus-1 (HSV-1) ICP27 open reading frame comprising a deletion of non-essential infected-cell protein 27 (ICP27) elements. In one aspect, the cell grows in suspension. In another aspect, the non-essential ICP27 elements are the 5′ and 3′ untranslated regions (UTRs).

The present disclosure is also directed to a cell line comprising a BHK cell described herein.

The present disclosure is also directed to a method of producing recombinant adeno-associated virus (rAAV) vectors comprising introducing recombinant herpes virus (rHSV) vectors containing AAV rep and cap sequences and sequence encoding a gene of interest into a BHK cell or cell line described herein; and culturing the cell or cell line under conditions to produce the rAAV vectors.

The present disclosure is also directed to a Chinese hamster ovary (CHO) cell that is adapted to grow in serum-free conditions, wherein the cell stably expresses one or more polypeptides necessary for entry and infection with herpes simplex virus-1 (HSV-1). In one aspect, the CHO cell stably expresses herpes virus entry mediator (HVEM) and/or Nectin-1. In another aspect, the HVEM and/or nectin-1 sequence has been codon optimized for expression in CHO cells.

The present disclosure is also directed to a CHO cell described herein.

The present disclosure is also directed to a method of producing recombinant adeno-associated virus (rAAV) vectors comprising introducing recombinant herpes virus (rHSV) vectors containing AAV rep and cap sequences and sequence encoding a gene of interest (GOI) into a CHO cell or cell line described herein; and culturing the cell or cell line under conditions to produce the rAAV vectors. In another aspect, the rHSV vectors are introduced at a multiplicity of infection of about 4:1, 6:1, 8:1, or 10:1 rHSV-rep/cap:rHSV-GOI. In another aspect, the AAV serotype is AAV6, AAV8 or AAV9. In another aspect, the gene of interest encodes any therapeutic biologic compound. In another aspect, the therapeutic biologic compound is an antibody or a chimeric antigen receptor.

### BRIEF DESCRIPTION OF THE DRAWINGS

FIG. 1 shows the plasmid constructions used for stable transfection and generation of eight pools. FIG. 1a shows four individual cassettes; Pool 1 (CMV-HVEM) in which HVEM ORF was constructed under CMV promoter and upstream SV40 poly A. Pool 2 (CMV-HVEM-CO) in which codon-optimized HVEM ORF was constructed under CMV promoter and upstream SV40 poly A. Pool 3 (CMV-Nectin-1) in which Nectin-1 ORF was constructed under CMV promoter and BGH poly A. Pool 4 (CMV-Nectin-1-CO) in which codon-optimized Nectin-1 ORF was constructed under CMV promoter and BGH poly A. FIG. 1b shows four dual cassettes; pool 5 (CMV-HVEM-Nectin-1) in which HVEM and Nectin-1 ORFs were constructed and flanked by CMV promoter and SV40 poly A and CMV promoter and BGH poly A, respectively. Pool 6 (CMV-HVEM-Nectin-1-CO) in which codon-optimized HVEM and Nectin-1 ORFs were constructed under a CMV promoter and upstream SV40 poly A and CMV promoter and upstream BGH poly A, respectively. Pool 7 (Spro-HVEM-Nectin-1) in which HVEM and Nectin-1 ORFs were constructed under a synthetic promoter (Spro) and upstream SV40 poly A and Spro and BGH poly A, respectively. Pool 8 (Spro-HVEM-Nectin-1-CO) in which codon-optimized HVEM and Nectin-1 ORFs were constructed and flanked by Spro and SV40 poly A and Spro and BGH poly A, respectively. FIG. 1c, shows high level of HVEM surface expression from pools (CMV-HVEM, CMV-HVEM-CO, CMV-HVEM-Nectin-1, CMV-HVEM-Nectin-1-CO, Spro-HVEM-Nectin-1 & Spro HVEM-Nectin-1-Co) was detected and expressed as mean fluorescence intensity (MFI). FIG. 1d shows high levelS of Nectin-1 surface expression from the pools (CMV-Nectin-1, CMV-Nectin-1-CO, CMV-HVEM-Nectin-1, CMV-HVEM-Nectin-1-CO, Spro-HVEM-Nectin-1 & Spro HVEM-Nectin-1-Co) was detected and expressed as MFI.

FIG. 2 shows green fluorescent protein (GFP) expression and rAAV9-GFP production from stable CHO cell pools. FIG. 2a shows the mean GFP expression from rHSV-nols-AAV-GFP infected stable cell pools (MOI=10) from A total six time points (12, 24, 36, 48, 72 and 96) post-infection. Pools 1 and 2 outperformed all other pools and host infected. All pools outperformed wildtype CHO host-infected (p<0.0001). FIG. 2b shows qPCR rAAV9-GFP titers (vg/mL) from supernatant of infected stable cell pools 24 hpi using MOI 1:1 of rHSV-nols-AAV-GFP and rHSV-AAV9 vectors. No significant difference was observed in rAAV9-GFP physical titers produced from eight tested pools. All co-infected cell pools produced higher rAAV9-GFP physical titers, compared to the wildtype host-infected cells. No significant difference in rAAV9-GFP titers was detected among tested pools (p=0.0976). All samples were tested in duplicate, and all data presented as mean±SD.

FIG. 3 shows generation of high and medium CHO-HVEM expressing clones for rHSV-GFP infection by flow cytometry analysis.

FIG. 4 shows characterization and testing of selected CHO-HVEM expressing clones for rHSV-1-GFP infection. FIG. 4a shows the final 24 selected recovered clones that had high HVEM expression (expressed as MFI). Ten of these clones were high HVEM expressing clones (clones 1, 7, 21, 23, 24, 33, 36, 40, 63 and 64). Other remaining 14 clones were medium HVEM expressing clones (clones 9, 11, 13, 14, 15, 16, 23, 28, 29, 42, 46, 51, 54, 62). FIG. 4b shows that all 24 clones were re-tested for rHSV-1 GFP vector entry and infection using IncuCyte (two time points were recorded). All infected clones showed high GFP expression post-infection. However, no significant difference in the GFP expression was observed among tested clones (p=0.5251).

FIG. 5 shows testing production of rAAV6.2-GFP in selected eight CHO-HVEM clones. FIG. 5a shows that cell viability drops post-rHSV-1 vectors co-infection. FIG. 5b shows viable cell density drops post-rHSV-1 vectors co-infection. FIG. 5c shows rAAV6.2-GFP titers from eight tested CHO-HVEM expressing clones using MOI 1:1. FIG. 5d shows testing rAAV6.2-GFP production in CHO-HV-C1 and CHO-HV-C62 clones using different MOIs. FIG. 5e shows testing rAAV8-GFP and rAAV9-GFP production in CHO-HV-C1 using MOI 4:1.

FIG. 6 shows significant improvement for both cell viability (FIG. 6a) and production of rAAV6.2GFP vector (FIG. 6b) following CHO-HV-C1 co-infection.

FIG. 7 shows a schematic of the process of harvesting and purifying rAAV vectors using a PEG-chloroform method.

FIG. 8 shows the analytical characterization of rAAVs produced in CHO-HV-C1 clone. FIG. 8a shows good expression of VP1, VP2 and VP3 capsid proteins from either purified rAAV6.2-GFP (produced using different MOIs; 4:2, 6:2, 8:2 and 10:2) and rAAV9-GFP vectors (produced using different MOIs; 4:2, 6:2 and 8:2). FIG. 8b shows minitransmission electron micrograph (miniTEM) of purified rAAV6.2-GFP showing 91% full capsids with no aggregates nor cellular debris. FIG. 8c shows miniTEM of purified rAAV9-GFP showing 79.5% full capsids. White arrows are used to refer to full capsids, while black arrows are used to refer to non-full (empty) capsids. FIG. 8d shows rAAVs capsid ratio detection using a CE-SDS method and shows absorbance values for VP1, VP2 and VP3 from tested rAAV6.2-GFP and rAAV9-GFP produced in CHO-HV-C1 clone with detection of impurities.

FIG. 9 shows testing infectious rHSV-1 residues in purified rAAVs on V27 cells. Complementing V27 cells that stably express HSV-1 ICP27 protein were tested for any infectious residues of rHSV-1 vectors used in production of rAAVs in CHO-HV-C1 clone. No cytopathic effect (CPE) appeared in wells inoculated with either PEG-Chloroform purified rAAV6.2-GFP and/or rAAV9-GFP vectors 2-4 days post infection, indicating complete inactivation of rHSV-1 vectors used for co-infection, compared to well inoculated with rHSV-AAV-GFP vector where clear CPE appeared to inform cell rounding and detachment of cell sheet.

FIG. 10 shows infectivity of rAAVs produced in CHO-HV-C1 cells. FIG. 10a shows infectivity of purified rAAV6.2-GFP vector produced in CHO-HV-C1 clone and purified with PEG-Chloroform method and tested on Ad293 cells and compared to infectivity of rAAV6.2-GFP vector produced in HEK293 cells and purified with chromatographic method. FIG. 10b shows infectivity of PEG-Chloroform purified rAAV9-GFP vector produced in CHO-HV-C1 clone and tested on Ad293 cells and compared to infectivity of rAAV9-ZsGreen vector produced in HEK293 cells and purified with chromatographic method FIG. 10c shows in vitro transduction of rAAVs produced in CHO-HV-C1 cells. Transduction of PEG-Chloroform purified rAAV6.2 and rAAV9-GFP produced in CHO cells were compared to those of rAAV6.2-GFP and rAAV9-ZsGreen produced in HEK293 cells and purified with chromatographic method.

FIG. 11 shows biodistribution of rAAVs-derived from CHO cells. FIG. 11a shows the experimental design. The mice were divided into five groups. G1, G2, G3, G4 were inoculated with 10¹¹ vg/100 μL per mouse of rAAV6.2-GFP-CHO, rAAV9-GFP-CHO, rAAV6.2-GFP from triple transient transfection (TTT) or rAAV9-ZsGreen from TTT, respectively. G5 was inoculated with sterile PBS. All mice were euthanized three weeks post tail vein injection and tissues (liver, heart, lung, kidney and skeletal muscle) were harvested and tested for GFP copies by qPCR and expression by confocal microscope. FIG. 11b shows GFP titers from G1 and G3 tissues using qPCR. FIG. 11c shows GFP/ZsGreen titers from G2 and G4 tissues using qPCR. FIG. 11d shows GFP expression from liver sections from all groups using confocal microscope.

FIG. 12 shows development and selection of HSV-1 producer CHO-ICP27 pools and clones. FIG. 12a shows development of stable CHO-HV1-ICP27 pools using random integration. The Chinese hamster codon-optimized HSV-1 ICP27 ORF was subcloned into an in-house plasmid for cell line development under the CMV promoter and upstream SV40 poly A. The same plasmid encoded puromycin cassette consisted of puromycin ORF downstream CMV promoter and BGH poly A. FIG. 12b shows development of stable CHO-HV1-ICP27 pools using CRISPR/Cas9 technology. Donor plasmid was constructed using an in-house pCLD plasmid backbone and encoded codon-optimized Chinese hamster ICP27 ORF downstream CMV promoter and upstream SV40 poly A and puromycin cassette flanked by SV40 promoter and SV40 poly A. The two cassettes were flanked by right and left homology arms (750 base pairs each). The total length of two cassettes is 4.1 kb. FIG. 12c shows mean fluorescence intensity (MFI) of ICP27 expression from final selected clones. Seven clones were site-integrated such as C6-S and 17 random-integrated clones such as C-11R.

FIG. 13 shows production of rHSV-AAV9 in CHO-HV1-ICP27-C11. Three different MOIs were used to test rHSV-AAV9 production in selected CHO-HV1-ICP27-C11 clone at 33° C. using an in-house medium. The harvested rHSV-AAV9 was titrated by plaque assay on V27 cells.

FIG. 14 shows construction of BHK-21-ICP27 expressing pools. FIG. 14a shows pool 1 that is expressing codon optimized ICP27 ORF under HSV-1 ICP27 endogenous promoter with puromycin as a selection marker. FIG. 14b shows pool 2 that is expressing codon optimized ICP27 ORF under the CMV promoter with puromycin as a selection marker. FIG. 14c, shows pool 3 that is expressing non codon optimized ICP27 ORF under the CMV promoter with neomycin as a selection marker.

FIG. 15 shows production of HSV-AAV6.2 in BHK-21-ICP27 pools. Recovered BHK21-ICP27 pools were tested for rHSV-AAV6.2 production (MOI 0.15 PFU/mL) using Xell HEK TF in a serum-free or in presence of 4% FBS. On day 3 post-infection, purified viruses were titrated by plaque assay on V27 cells.

FIG. 16 shows silver staining of impurities from PEG-Chloroform purification of rAAVs samples.

### DETAILED DESCRIPTION

Recent studies have shown that production of different recombinant adeno-associated viruses (rAAV) using recombinant Herpes Simplex Virus 1 (rHSV-1) vectors produced rAAV with higher physical and transduction titers compared to those produced via the commonly used triple transient transfection method and/or baculovirus-based system. However, rAAV-based HSV production platform currently faces two major challenges: (1) reliance on serum-supplemented commercial medium for high productivity, leading to costly large-scale production and high risk of introduction of adventitious agents into the final product, and (2) challenging to scale-up rHSV-1 vectors in adherent Vero cells expressing HSV-1 ICP27 protein (such as the V27 cell line). To address the first challenge, the present disclosure provides eight serum-free-adapted CHO cell pools express receptors (HVEM and/or Nectin-1) essential for HSV-1 entry and infection. Using high-throughput methods, the present disclosure provides a top HVEM receptor expressing clone (called CHO-HV-C1). Interestingly, higher yields of rAAV6.2-GFP, rAAV8-GFP and rAAV9-GFP vectors were achieved with lower Multiplicity of Infection (MOI) with titers ~9.21 log₁₀, 9.40 log₁₀ and 9.61 log₁₀ viral genomes/mL (vg/mL), respectively, produced within 24 hours post co-infection in CHO-HV-C1 clone, compared to non-CHO-based platforms that reported 10 log₁₀ vg/mL in 48-72 hours post co-infection with higher MOI (Kang et al., 2009. Gene Therapy 16, 229-239). Moreover, rAAV produced in CHO-HV-C1 clone had comparable in vitro and in vivo transduction potency to those produced by triple transient transfection. To address the second challenge, the present disclosure provides in-house, serum-free, suspended BHK-21 cell pools engineered to express HSV-1 ICP27 protein. Interestingly, BHK-21-ICP27 expressing pools grown in serum-free medium produced comparable rHSV-I titers to V27 cells.

Gene therapy has very promising potential for the treatment of many different diseases for which no available treatments currently exist, such as cystic fibrosis, heart failure, and Duchenne muscular dystrophy. In recent years, gene therapy has focused on the use of rAAV vectors because of their long-lasting expression after delivery to target organs and their non-pathogenic profiles in humans. Current manufacturing methods for rAAVs for clinical studies include triple transient transfection (the most common method), packaging or producer cell lines, and helper virus systems, such as those using HSV-1, baculovirus, and/or human adenovirus-5. Transfection-based methods such as triple transient transfection are difficult to scale-up and result in low specific yields of infectious particles (ip) and DNase-resistant particles (Grimm et al., 1998. Hum Gene Ther; 9: 2745-2760; Zolotukhin et al., 2002. Methods; 28:158-167). On the other hand, packaging or producer cell lines are limited to the production of one rAAV serotype/product. Recent studies showed that rHSV-1-assisted rAAV production provides a highly-efficient manufacturing method (Kang et al., 2009). However, typical manufacturing of rAAV using the rHSV-1 system involves infection of virus production cells, such as BHK-21 cells, with two replication deficient rHSV-1 vectors in a serum-supplemented medium, which is very expensive for large-scale production and may introduce adventitious viral agents and/or prions into the final product. Another drawback of manufacturing of rAAV using the rHSV-1 system is the challenge of rHSV-1 vectors stock scale-up because current rHSV-1 vectors stock production depends on using adherent Vero cells expressing HSV-1 ICP27 protein (called V27 cells, Rice and Knipe J Virol. 1990 April; 64(4):1704-15).

CHO cells are the predominant mammalian cell type used to produce recombinant protein biologics because of their ability to correctly fold, assemble, and modify recombinant proteins (Aggarwal, 2014. Nat. Biotechnol. 32, 32-39; Jayapal et al., 2007. Cell Engineering Progress, 103, 40-47; and Walsh, 2018. Nat. Biotechnol; 36:1136-1145). Certain animal cell types, such as swine testis (ST) and CHO cells, can bind HSV-1 virus efficiently but restrict the viral entry (Shieh et al., 1992. J. Cell Biol. 116, 1273-1281; Subramanian et al., 1994. J. Virol. 68, 5667-5676). In addition, it has been reported that swine and CHO cells become susceptible to HSV-1 entry upon expression of human cDNA encoding HVEM (Montgomery et al, 1996. Cell Vol. 87, 427-436). The present disclosure aimed to engineer suspension, serum-free-adapted CHO cells to produce different rAAVs using the HSV-1 helper system, a virus which naturally does not infect wild-type CHO cells.

rAAVs are the leading platform for gene delivery with three licensed products approved by the end of 2021 (Bulcha et al., 2021). rAAV have several advantages as gene delivery vectors, including their ability to transduce a variety of proliferating and non-proliferating cells, accommodate cell/tissue-specific promoters, and elicit diminished immune response in comparison to other viral vectors (Kang et al., 2009).

Currently, there are different cell culture expression platforms for rAAVs, including stable packaging cell lines, which express the rep and the cap genes of a desired rAAV serotype, stable proviral cell lines that stably express Rep, Cap, and the transgene, and triple transient transfection (Clark et al, 1995. Hum Gene Ther; 6: 1329-1341, Clark, 2002. Kidney Int; 61s: 9-15, Qiao et al, 2002. Kidney Int; 61s: 9-15). The triple transient transfection is the most common method for production of rAAVs, which uses three plasmids: one encoding the gene of interest (GOI) flanked by the AAV inverted terminal repeat (ITR), a second encoding the rep and cap genes of AAV, and the third encoding the adenovirus helper function genes. This three-plasmid system can be simplified to a co-transfection by inclusion of the helper genes in the rep-cap plasmid (Grimm et al., 1998 and Clark K. R., 2002). However, triple transient transfection method is challenging to scale-up for very high doses resulting in low specific yields of infectious particles (ip), and often result in high DNase-resistant particles (DRP/ip) ratios (50-100) (Grimm et al., 1998. Hum Gene Ther; 9: 2745-2760; Zolotukhin et al., 2002. Methods; 28:158-167).

Like adenovirus, it has been reported that recombinant HSV-1 (rHSV-1) can also support rAAV replication as part of a helper virus system (Buller et al, 1981). The minimum HSV helper genes required for rAAVs replication are the HSV helicase primase complex (UL5, UL8, UL52), the HSV DNA polymerase, and the HSV DNA-binding protein (UL29) genes (Weindler and Heilbronn, 1991. J Virol; 65: 2476-2483). Moreover, it has been reported that rHSV co-infection method that used two HSV-1 vectors, one encoding AAV2 rep and cap of the desired AAV and the second encoding the GOI, generated very high specific yields of rAAVs of multiple different serotypes, with a very low (DRP/ip) ratio, in various cell lines (Kang et al, 2009). Another advantage for HSV helper system is that HSV is capable of replicating and providing helper functions in different mammalian cells which means HSV helper functions bypass the host range restriction for rAAVs production, compared to adenovirus helper-assisted rAAV production that requires human cell lines (Buller et al., 1970. J Gen Virol; 43: 663-672).

Chinese hamster ovary (CHO) cells are predominantly used as expression hosts for recombinant monoclonal antibody (mAb) and therapeutic protein production, which comprises the fastest growing segment of the biopharmaceutical industry (Walsh G., 2018). Due to the regulatory acceptability and low manufacturing cost of CHO cells relative to human cells, using CHO cells for viral production is desirable. However, there is limited experience, if any, in literature about the use of CHO cells for rAAV production, likely due to cellular restriction factors in these cells that may affect rAAV production and interfere with virus packaging. For example, it has been shown that CHO cells did not support vaccinia virus replication at the stage of viral intermediate protein synthesis (Ramsey-Ewing and Moss. Virology. 1995 Feb. 1; 206(2):984-93). In addition, CHO cells are naturally resistant to HSV-1 productive infection because of the lack of key receptors for infection with non-replicating HSV-1 (Montgomery et al., 1996. Cell Vol. 87, 427-436). The present disclosure provides serum free-adapted suspension CHO-CAT-S cells engineered to be permissive for HSV-1 entry and infection for the production of diverse rAAV using the HSV-1 system.

### Definitions

In order that the present disclosure can be more readily understood, certain terms are first defined. As used in this specification, except as otherwise expressly provided herein, each of the following terms shall have the meaning set forth below. Additional definitions are set forth throughout the specification.

It is to be noted that the term “a” or “an” refers to one or more of that entity; for example, “a feed medium,” is understood to represent one or more feed media. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “adeno-associated virus”, “AAV virus”, “AAV virion,” “AAV viral particle,” and “AAV particle”, used as synonyms herein, refer to a viral particle composed of at least one capsid protein of AAV and an encapsulated polynucleotide corresponding to the AAV genome. The wild-type AAV refers to a virus that belongs to the genus Dependovirus, family Parvoviridae. The wild-type AAV genome is approximately 4.7 kb in length and consists of a single stranded deoxyribonucleic acid (ssDNA) that can be positive or negative-sensed. The wild-type genome includes inverted terminal repeats (ITR) at both ends of the DNA strand, and three open reading frames (ORFs). The ORF rep encodes four Rep proteins necessary for AAV lifecycle. The ORF cap contains nucleotide sequences encoding capsid proteins VP1, VP2 and VP3, which interact to form a capsid of icosahedral symmetry. Finally, the assembly-activating protein (aap) ORF, which overlaps with the cap ORF, encodes for the AAP protein that appears to promote capsid assembly. If the particle comprises a heterologous polynucleotide (i.e., a polynucleotide different from a wild-type AAV genome, such as a transgene to be delivered to a mammalian cell) flanked by AAV ITRs, then it is typically known as “AAV vector particle” or “AAV viral vector” or “AAV vector” or “recombinant AAV vectors”. The invention also encompasses the use of double stranded AAV or self-complimentary AAV, also called dsAAV or scAAV.

An “AAV virus” or “AAV viral particle” refers to a viral particle composed of at least one AAV capsid protein (preferably by all of the capsid proteins of a wild-type AAV) and an encapsidated polynucleotide. If the particle comprises a heterologous polynucleotide (i.e., a polynucleotide other than a wild-type AAV genome, such as a transgene to be delivered to a mammalian cell), it is typically referred to as an “rAAV vector particle” or simply an “rAAV vector”.

“Packaging” refers to a series of intracellular events that result in the assembly of the capsid proteins and encapsidation of the vector genome to form an AAV particle.

AAV “rep” and “cap” genes refer to polynucleotide sequences encoding replication and encapsidation proteins of adeno-associated virus, respectively. They have been found in all AAV serotypes examined and are described below and in the art. AAV rep and cap are referred to herein as AAV “packaging genes”.

The term “hybrid AAV” as used herein refers to an AAV comprising a capsid protein of one AAV serotype and genomic material from another AAV serotype.

The term “chimeric AAV” as used herein refers to an AAV that comprises genetic and/or protein sequences derived from two or more AAV serotypes, and can include mutations made to the genetic sequences of those two or more AAV serotypes. An exemplary chimeric AAV can comprise a chimeric AAV capsid, for example, a capsid protein with one or more regions of amino acids derived from two or more AAV serotypes.

The term “AAV variant” as used herein refers to an AAV comprising one or more amino acid mutations in its genome or proteins as compared to its parental AAV, e.g., one or more amino acid mutations in its capsid protein as compared to its parental AAV.

The term “viral vector” refers to a gene transfer vector or a gene delivery system derived from a virus. Such vector can be constructed using recombinant techniques known in art. In some respects, the virus for deriving such vector is selected from AAV, helper-dependent adenovirus, hybrid adenovirus, Epstein-Bar virus, retrovirus, lentivirus, herpes simplex virus, hemaglutinating virus of Japan (HVJ), Moloney murine leukemia virus, poxvirus, and HIV-based virus.

The term “AAV virion” or “AAV particle,” as used herein refers to a virus particle comprising a capsid comprising at least one AAV capsid protein that encapsidates an AAV vector as described herein, wherein the vector can further comprise a heterologous polynucleotide sequence or a transgene in some embodiments.

The term “engineered cell” and its grammatical equivalents as used herein refers to a cell comprising at least one alterations of a nucleic acid within the cell's genome or comprising at least one exogenous nucleic acid or protein. Alterations include additions, deletions, and/or substitutions within a nucleic acid sequence. As such, engineered cells, include cells that contain an added, deleted, and/or altered gene.

Various aspects of the disclosure are described in further detail in the following subsections.

### Adeno-Associated Virus (AAV)

Adeno-Associated Virus (AAV) is a non-pathogenic, single-stranded DNA parvovirus. AAV has a capsid diameter of about 20 nm. Each end of the single-stranded DNA genome contains an inverted terminal repeat (ITR), which is the only cis-acting element required for genome replication and packaging. The AAV genome carries two viral genes: rep and cap. The virus utilizes two promoters and alternative splicing to generate four proteins necessary for replication (Rep78, Rep 68, Rep 52, and Rep 40). A third promoter generates the transcript for three structural viral capsid proteins, 1, 2 and 3 (VP1, VP2 and VP3), through a combination of alternate splicing and alternate translation start codons (Berns K I et al., Bioessays. 1995; 17:237-45). The three capsid proteins share the same C-terminal 533 amino acids, while VP2 and VP1 contain additional N-terminal sequences of 65 and 202 amino acids, respectively. The AAV virion contains a total of 60 copies of VP1, VP2, and VP3 at a 1:1:5 that had been seen in crude extracts (Aucoin M G et al., Biotechnol Adv. 2008; 26(1):73-88) or at a range of 1:1:8 to 1:1:20 by densitometry (Grimm D et al., Gene Ther, 1999; 6(7):1322-330; Kronenberg S et al., EMBO Rep. 2001; 2(11):997-1002) arranged in a T=1 icosahedral symmetry (Rose J A et al. J Virol. 1971; 8:766-70). AAV requires Adenovirus (Ad), Herpes Simplex Virus (HSV) or other viruses as a helper virus to complete its lytic life cycle (Atchison R W et al., Science. 1965; 149:754-6; Hoggan M D et al., Proc Natl Acad Sci USA. 1966; 55:1467-74). Moreover, in the absence of the helper virus, Wild-type (wt) AAV establishes latency by integration with the assistance of Rep proteins through the interaction of the ITR with the chromosome (Berns et al., 1995).

### AAV Serotypes

There are a number of different AAV serotypes, including but not limited to AAV1, AAV2, AAV3, AAV4, AAV5, AAV6, AAV7, AAV8, AAV9, and rh-AAV-10. In vivo studies have shown that the various AAV serotypes display different tissue or cell tropisms. For example, AAV1 and AAV6 are two serotypes that are efficient for the transduction of skeletal muscle (Gao G P et al., Proc Natl Acad Sci USA. 2002; 99:11854-11859; Xiao W et al., J Virol. 1999; 73:3994-4003; Chao H et al., Mol Ther. 2000; 2:619-623).

Since the development of naturally-occurring AAV serotypes into gene therapy vectors, much effort has been focused towards understanding the tropism of each serotype so that further modification to the virus could be performed to enhance the efficiency of gene transfer. One approach is to swap domains from one serotype capsid to another and, thus, create hybrid vectors with desirable qualities from each parent. As the viral capsid is responsible for cellular receptor binding, the understanding of viral capsid domain(s) critical for binding is important. Mutation studies on the viral capsid (mainly on AAV2) performed before the availability of the crystal structure were mostly based on capsid surface functionalization by adsorption of exogenous moieties, insertion of peptide at a random position, or comprehensive mutagenesis at the amino acid level (Choi et al. Curr Gene Ther. 2005 June; 5(3): 299-310).

In some aspects, the disclosure provides a method for producing rAAV particles with capsid proteins expressed by multiple serotypes of AAV. This is achieved by co-infection of producer cells with an rHSV expression virus and with an rHSV-rep2capX helper virus in which the cap gene products are derived from serotypes of AAV other than, or in addition to, AAV2. Recombinant AAV vectors have generally been based on AAV2 capsids. It has recently been demonstrated that rAAV vectors based on capsids from AAV1, AAV3, AAV4, AAV5, AAV8 or AAV9 serotypes differ from AAV2 in their tropism.

Capsids from other AAV serotypes offer advantages in certain in vivo applications over rAAV vectors based on the AAV2 capsid. First, the appropriate use of rAAV vectors with particular serotypes may increase the efficiency of gene delivery in vivo to certain target cells that are poorly infected, or not infected at all, by AAV2 based vectors. Secondly, it may be advantageous to use rAAV vectors based on other AAV serotypes if re-administration of rAAV vector becomes clinically necessary. It has been demonstrated that re-administration of the rAAV vector with the same capsid can be ineffective, possibly due to the generation of neutralizing antibodies generated to the vector. This problem may be avoided by administration of an rAAV particle whose capsid is composed of proteins from a different AAV serotype not affected by the presence of a neutralizing antibody to the first rAAV vector. It will be recognized that the construction of recombinant HSV vectors similar to rHSV but encoding the cap genes from other AAV serotypes (e.g., AAV1, AAV2, AAV3, AAV5 to AAV9) is achievable using the methods described herein to produce rHSV. In certain aspects, recombinant AAV vectors constructed using cap genes from different AAVs are performed.

### EXAMPLES

### Experimental Methods

### Generation of Stable CHO Pools

The open reading frames (ORFs) of human HVEM and Nectin-1 were downloaded from NCBI database (GenBank U70321.1 and AF060231.1, respectively). Both HVEM and Nectin-1 ORFs were codon-optimized for expression in hamster cells using online tools (https://www.idtdna.com/CodonOpt and https://www.thermofisher.com/us/en/home/life-science/cloning/gene-synthesis/geneart-gene-synthesis/geneoptimizer.html, respectively). The delivered plasmids encoding either HVEM or Nectin-1 were sub-cloned into an in-house plasmid downstream of the enhanced human cytomegalovirus (CMV) promoter and/or synthetic promoter (Brown et al., 2017) generating eight different constructs. All constructed plasmids encoded glutathione synthetase (GS) under the control of the SV40 promoter allowing for the selection of transfected cells in methionine sulfoximine (Bebbington et al, 1992. Bio/technology (Nature Publishing Company), 10 (2), 169-175). All final plasmids were verified by whole plasmid sequencing (Macrogen). Proprietary, in-house, suspension, serum-free-adapted CHO cells were thawed into a 125 mL shake flask and cultured in 30 mL CD-CHO medium (Thermo Fisher), supplemented with 6 mM L-glutamine (Gibco), dextran sulphate (50 mg/mL, Sigma-Adrich) and incubated in a 6% CO₂ humidified incubator at 37° C., 120 rpm agitation. Viable cell density (VCD) and viability were measured daily using a Vi-Cell automated cell counter (Beckman Coulter).

Stable CHO cell pools were generated according to a standard in-house protocol. In brief, eight aliquots of CHO CAT-S cells (1×10⁷ viable cells per aliquot) were pelleted for 5 minutes at 200×g. Cell pellets were mixed with 7 μg of each purified linearized in-house pCLD plasmid and were then transfected using Amaxa cell line nucleofector Kit V (Lonza) according to the manufacturer's instruction. After 24 hours of transfection, cell viability was measured, and 75 μM/mL MSX (Sigma-Aldrich) was added to each pool for recombinant cell selection. Aliquots (1×10⁶ viable cells per pool) from recovered cell pools were tested for either HVEM and/or Nectin-1 receptor expression using FACS staining. In brief, cells were incubated with 150 μL of 1:200 diluted either anti-CD270 (HVEM) eBioscience PE clone eBioHVEM-122 (Invitrogen,) and/or 1:200 diluted Nectin-1 monoclonal antibody clone R1.302-PE (Invitrogen) in PBS (Gibco) supplemented with 1% bovine serum albumin (BSA) (Invitrogen) for 15 minutes at room temperature in dark. After incubation, diluted antibodies were poured off and cells were then fixed with Fix and Perm medium A (Life Technologies) and incubated for 15 minutes at room temperature in the dark. The stained cells were then washed twice with PBS and then resuspended in FACS buffer (PBS supplemented with 0.1% BSA) for flow cytometry using LSR II instrument (BD Biosciences). Flow data was analyzed with FlowJo v10.0 software (Tree Star, Inc).

rHSV-1 Infection in Stable CHO Pools

An aliquot (1×10⁶ viable cells) from each recovered cell pool and CHO CAT-S host cells were infected with rHSV-GFP (MOI=10) in 6-well cell culture plate (Corning) for 1 hour for virus adsorption at 37° C. in humidified 5% CO₂ incubator. After 1 hour, infected cell pools were centrifuged for 5 minutes at 1200 rpm and the excess virus supernatant was discarded. Infected cell pellets were resuspended into CD-CHO medium and incubated at 37° C. in 5% CO₂ humidified incubator connected with the IncuCyte (Sartorius). Cell imaging for GFP expression from infected pools was determined using the IncuCyte default settings, and GFP expression was captured every 12 hours from each infected cell pool for total five time points.

rAAV9-GFP Vector Production in Stable CHO Pools

An aliquot of each cell pool (1×10⁶ viable cells) was infected with MOI 1:1 from rHSV-AAV9 rep/cap:rHSV-AAV-GFP vectors into 6-well culture plates (Corning). Infected cell pools were incubated for 24 hours at 37° C. in 5% CO₂ humidified incubator. After 24 hours, the infected cell supernatant was collected and tested for rAAV9-GFP titer using qPCR. In brief, collected supernatant was digested at 37° C. for 1 hour with DNase I (200 U/μL, Invitrogen) followed by 10-minute incubation at 95° C. for enzyme inactivation. The digested samples were then incubated with an equal volume of proteinase K digestion mix (200 mM NaCl, 20 mM Tris-HCl, pH 8.0, 2 mM ethylenediaminetetraacetic acid, pH 8.0; 0.5% sodium dodecyl sulfate and proteinase K (20 mg/mL) at 55° C. for 1 hour followed by enzyme inactivation for 10 minutes at 95° C. The reaction for absolute qPCR quantification was done using PCR thermocycler (QuantaBio Q, Qiagen) using an in-house standard linearized AAV plasmid in a 20 μL reaction contained TaqMan Fast Universal PCR 2× Master Mix (Applied Biosystems) and 20 μM of each CMV-Forward primer (5′-TTCCTACTTGGCAGTACATCTACG′-3), CMV-Reverse primer (5′-GTCAATGGGGTGGAGACTTGG-′3) and of CMV probe (5′-FAM-TGAGTCAAACCGCTATCCACGCCCA-NFQ-′3), in addition to 5 μL of diluted template. The PCR cycling profile was 95° C. for 2 minutes and 40 cycles of 95° C. for 5 seconds and 60° C. for 30 seconds.

Generation and Testing rHSV-GFP Infection in CHO-HVEM Expressing Clones

Single-cell deposition cloning was performed using a BD Influx cell sorter (BD Biosciences) according to Evans et al., 2015 (Biotechnology Progress, 31(5), 1172-1178). In brief, an aliquot containing 3×10⁶ viable HVEM CHO expressing cells were stained with 1:200 PBS diluted anti-human CD270 (HVEM)-PE (Invitrogen) antibody for 15 minutes in the dark at room temperature. Stained cells were washed twice with sterile PBS, pelleted at 200×g for 5 minutes and then resuspended in 1 mL sorting buffer. Cells were sorted from the PE-gated fraction into two 384-well plates (Agilent) containing an in-house conditioned medium supplemented with 50 μM MSX/mL. The selected recovered clones were further tested rHSV-GFP infection and the mean GFP expression was calculated from two time points (24 and 48) hpi using IncuCyte as previously described.

Production of rAAV6.2-GFP Vector in Final CHO-HVEM Clones

The selected clones were also further tested for rAAV6.2-GFP vector production using an MOI of 1:1 for rHSV-AAV6.2 rep/cap:rHSV-GFP. All infected clones were incubated at 37° C. in a 5% CO₂ humidified incubator with 120 rpm agitation for three days. An aliquot (1 mL) from each co-infected clone was harvested at 24 and 48 hours post co-infection and centrifuged at 200×g for 5 minutes. The harvested samples (cell pellets) were then prepared for rAAV titration using qPCR. In brief, infected cell pellet was collected, mixed with the AAV lysis buffer (50 mM Tris, pH 8.0, 150 mM NaCl), and subjected to three cycles of freeze/thawing in an isopropanol dry ice bath followed by centrifugation at 12,000 rpm for 30 minutes at 4° C. After centrifugation, the supernatant was collected and the rAAV6.2-GFP titers were determined using qPCR as previously described. In a subsequent experiment, two of the final selected clones were further tested for rAAV6.2-GFP vector production using different MOIs and incubation temperature. The rAAV6.2-GFP titer was determined 24 h post co-infection from harvested cell pellet and titrated using qPCR as previously described. Purification of rAAVs produced in final selected CHO clone was done according to (Negrini et al., Curr Protoc Neurosci. 2020 September; 93(1): e103). rAAVs produced in the final selected CHO clone was purified using the polyethylene glycol (PEG) chloroform method according to Negrini et al., with some modifications (Negrini et al., 2020), including using 0.5% Triton X-100 (v/v) for cell lysis and rHSV-1 inactivation, followed by precipitation using polyethylene 8% glycol8000 and 150 mM NaCl. Viral pellets were further treated with Benzonase (Sigma; 50 U/mL) and RNase (Invitrogen; 10 μg/mL) at 37° C. for 1 hour followed by 1:1 chloroform (Sigma) treatment. The aqueous layer post chloroform treatment was collected and concentrated using AMICON filters (Sigma). The concentrated viruses were stored at −80° C. until used.

Analytical Characterization of CHO-Derived rAAVs

Purified rAAVs were tested for capsid protein expression using Western blotting. In brief, purified rAAV6.2-GFP and rAAV9-GFP vectors produced in the final selected clone using different MOIs of rHSV-1 vectors were prepared for SDS-PAGE gel by adding the appropriate volume of 4× NuPAGE lithium dodecyl sulfate (LDS) sample buffer (Life Technologies) and 10× NuPAGE sample-reducing agent (Thermo Fisher). The samples were then incubated at 70° C. for 10 minutes. Equal volumes of rAAVs were loaded on a Bolt 4-12% Bis-Tris plus 12-well gel (Invitrogen) for each serotype and were run using 1× NuPAGE running buffer (Thermo Fisher). After the run, the gel was subjected to dry transfer using an iBolt 2NC mini stack (Invitrogen) followed by blocking in 5% skimmed milk (Amresco) diluted in TBS (Bio-Rad) for 1 hour at room temperature. After blocking, membrane was incubated with 1:200 AAV VP1, VP2, VP3 5% skimmed milk diluted antibody (Genentech, USA) overnight at 4° C. with gentle shaking. After incubation, the blotted membrane was washed three times with TBS supplemented with 0.1% Tween (Life Technologies) and then incubated with 1:100 goat anti-mouse IgG (Thermo Scientific) for 1 hour followed by washing. The membrane was incubated with Supersignal West Pico Plus Substrate (Thermo Scientific) before image detection using Amersham Imager 680 (GE Healthcare). In a subsequent experiment, the purified rAAVs from the final selected CHO clone were visualized using a mini-transmission electron microscopy (Mini-TEM; Vironova). In brief, PEG-chloroform purified rAAV6.2-GFP and rAAV9-GFP samples were placed on a 400-mesh glow-discharged carbon grid by first inverting the grid and placing it on top of a 10 μL droplet of rAAV, deposited on parafilm, for 30 seconds. Excess sample was blotted off by gently touching the edge of the grid against a Whatman filter paper. The grid was then washed twice with two 20 μL droplets of double distilled water. The sample-containing grid was then stained with a 20 μL droplet of 1.5% uranyl acetate for 10 seconds. Excess stain was blotted off by gently touching the edge of the grid against a Whatman filter paper. rAAV samples were then visualized using a Mini-TEM instrument. In another separate experiment, rAAVs capsid protein (VP1:VP2:VP3) ratio analysis was performed using an in-house developed capillary electrophoresis sodium dodecyl sulfate (CE-SDS) method according to Kurasawa et al. (Mol Ther Methods Clin Dev. 2020 Oct. 4; 19:330-340).

Infectious rHSV-1 Residues from Purified CHO-Derived rAAVs

V27 cells that express a stable copy of rHSV-1 ICP27 protein were seeded at 0.5×10⁶ viable cells/well in a 6-well culture plate in DMEM supplemented with 10% FBS (Gibco) and 500 μg/mL geneticin (Gibco) overnight. After 16 hours, the cells were washed twice with sterile PBS, and were then infected with purified rAAV6.2-GFP and rAAV9-GFP vectors at 1:100 dilution (~10¹⁰ vg/mL). The rHSV-AAV-GFP vector was used as positive control (MOI=0.15 PFU/cell). Infected cells were incubated for 2 hours at 37° C. for virus adsorption. After incubation, excess virus was removed and infection medium (DMEM supplemented with 2% FBS) was added, and the plate was incubated and monitored with the IncuCyte for 4 days for capturing any cytopathic effects.

Infectivity and In Vitro Transduction of CHO-Derived rAAVs

For infectivity, Ad293 cells were seeded in a 96-well culture plate (2×104 cells/well) and incubated at 37° C. in a humidified, 5% CO₂ incubator overnight. Ten-fold dilution of rAAV6.2-GFP, rAAV9-GFP and/or rAAV9-ZsGreen produced either in CHO clone and purified with the PEG-chloroform and/or produced in HEK293 cells using triple transfection (Kimura et al., Sci Rep 9, 13601, 2019) and purified with affinity chromatography were carried out. Each virus dilution was used to infect four wells, and infected cells were incubated at 37° C., 5% CO₂ incubator for 5 days. On day 5 post-infection, infected cells were imaged for GFP/Zs-Green expression using the IncuCyte default settings, and virus titers were calculated using the Reed and Muench method (Reed and Muench, American Journal of Epidemiology; 27; 3; 493-497, 1938. For transduction analysis, Ad293 cells were cultured as mentioned above and were then infected with different multiplicity of transduction (MOT) from CHO-derived rAAVs and/or similar vectors produced in HEK293 cells using the triple transfection as described above.

Biodistribution of CHO-Derived rAAVs

All animal experiments were approved by the Institutional Animal Care and Use Committee of AstraZeneca (Gaithersburg, MD, USA). Eight weeks old male C57bl/6 mice were purchased from Jackson Laboratory (Bar Harbor, ME). Mice were divided into five groups (n=5 per group) and all mice were inoculated with 1×10¹¹ vg/100 μL of the appropriate rAAV vector (or saline) via the tail vein using an insulin BD syringe (Becton Dickinson). The inoculated mice were monitored daily for any clinical symptoms of illness. Three weeks post injection, mice were euthanized via CO₂ and organs were harvested. A half section of the harvested tissues were frozen in dry ice in a microcentrifuge tube for qPCR analysis, and the other half were fixed in 10% neutral buffered formalin for histology work. DNA extraction from the harvested tissues was done using the All-Prep DNA/RNA Mini Kit (Qiagen) according to manufacturer's instructions. The qPCR reactions were carried out on the QuantStudio 7 Flex using in-house linearized plasmids: pAAV-GFP for groups 1, 2 and 3 and pAAV-ZsGreen for group 4. Extracted genomic DNA (100 ng) was used as a template, and specific GFP primers and probes (forward 5′-GAACCGCATCGAGCTGAA-‘3, Reverse 5′-TGCTTGTCGGCCATGATATAG-‘3 and probe 5′/56-FAM/ATCGACTTC/ZEN/AAGGAGGACGGCAAC/3IABkFQ ‘3 and ZsGreen primers and probe forward 5′-GTACCACGAGTCCAAGTTCTAC-‘3, reverse 5′-CACGTCGCCCTTCAAGAT-‘3 and probe 5′/56-FAM/CCCGTGATG/ZEN/AAGAAGATGACCGACAA/3IABkFQ/‘3). Cycling conditions were initial denaturation for 95° C. for 3 seconds, 40 cycles of 95° C. for 10 seconds, annealing/extension at 60° C. for 20 seconds. For histology, thin liver tissue sections from collected tissues were prepared using a Leica 3050s microtome (Germany), stained, mounted, and assessed for GFP detection using confocal microscopy. GFP quantification from liver slides was done using an axioscanner.

Engineering rHSV-1 Vector Producer Cell Pools

Two proprietary suspended cell lines; CHO-K1 and BHK-21 based, were selected for the engineering work. For suspended CHO cells, two strategies were used: (1) random integration, where HSV-1 ICP27 ORF (GenBank AB235845.1) sequence was codon-optimized for hamster cells expression by IDT, chemically synthesized, and was then sub-cloned downstream of the CMV promoter into an in-house plasmid encoding the puromycin cassette for selection. For site-integration, the exon1 from C120rf35 locus from CHO genome (GenBank XM_027430029) was selected as one of transcription hotspots (Zhao et al., Appl Microbiol Biotechnol. 2018 July; 102(14):6105-6117). The CRISPy bioinformatic tool with default parameters was used to select sgRNA target sequence according to (Ronda et al 2014). The selected gRNA target (5′GGACTTAACCACTCGATGGC-‘3) was synthesized by IDT and delivered as gblocks, annealed, and sub-cloned into the linearized CRISPR nuclease expression vector (GeneArt CRISPR CD4) backbone to generate sgRNA expression vectors using GeneArt CRISPR CD4 kit (Invitrogen) according to the manufacturer's instructions. The donor DNA plasmid was constructed using the backbone of an in-house plasmid, avoiding any protospacer adjacent motif (PAM) sites identical to the gRNA target. The 5′ and 3′ homology arms (750 base pairs each) flanking the sgRNA target sequence were chemically synthesized (IDT) with 110 nucleotides as genetic linker in between contained different restriction sites for cloning. Transfection of the final AAV CHO clone followed the process as previously described. Transfected cell pools were selected using 5 μg puromycin/mL for two weeks. Clones were generated via single-cell deposition using a BD Influx cell sorter (BD Biosciences) based on a previously described method (Evans et al., 2015) in 384-well plates using anti HUI CD270 (HVEM) eBioscience PE clone eBioHVEM-122. (2) for BHK-21 suspended cells, a propriety an in-house serum-free suspension adapted BHK-21 cells were maintained in Xell HEK TF medium (Xell AG, Germany). Cells were seeded at density of (0.3×10⁶ cells/ml) in 30 ml medium in 125 ml shake flask (Nunc, Denmark) and incubated in shaking incubator with agitation at 120 rpm, 5% CO₂ for three days. The ICP27 (ORF) from the HSV-1 genome (GenBank KM222723.1) was downloaded and codon-optimized using the online IDT tool (https://www.idtdna.com/CodonOpt), synthesized and delivered in a commercial plasmid. The codon-optimized ICP27 gene was subcloned into an in-house plasmid downstream either the CMV promoter or the endogenous ICP27 promoter, generating pCLD-CMV-ICP27 and pCLD-EN-ICP27, respectively using restriction enzyme cloning. The above constructed pCLD plasmids contained puromycin cassette for the selection. Another version of the ICP27 (non-codon-optimized) ORF was synthesized by the GeneArt (Thermo Fisher, USA), subcloned using BamHI-NotI restriction cloning into a commercial expression plasmid downstream the CMV promoter, generating the third plasmid (called pCDNA-ICP27). The pCDNA-ICP27 contained neomycin cassette for selection. The three plasmids were amplified in competent DH5 alpha and purified using maxiprep (Qiagen, USA). Final plasmids were confirmed by sanger sequencing (Macrogen, USA). Aliquots of BHK-21 cells (7×10⁶ each aliquot) were transfected with the linearized plasmids (2.5 μg each) using the Amaxa nucleofection Kit L (Lonza, USA) in nucleofector II according to the manufacturer's instructions. Forty-eight hours after transfection, transfected cells were put under either 400 μg/ml geneticin (Gibco, USA) and/or 10 μg puromycin (Gibco, USA) selection for three weeks.

rHSV-1 Infection in Selected CHO-ICP27 Clone

The final selected clone was tested for rHSV-1 infection. In brief, an aliquot (1×10⁶ viable cells) were infected with rHSV-AAV9 using (MOI=10) in antibiotic free DMEM and were then incubated for 20 minutes on ice followed by incubation at 37° C. for 2 hours. After incubation, excess virus was decanted, and the infected cells were washed twice with sterile PBS to remove any residual rHSV-AAV9 vectors. After washing, infected cells were covered with an in-house medium (2 mL/well) in 6-well plate and incubated at 37° C. in 5% CO₂ humidified incubator for 24 hours. After 24 hours, 1 mL supernatant from infected cells was collected and inoculated on V27 cells to determine cytopathic effect. Harvested viruses from inoculated V27 cells were purified by ultracentrifugation and subjected to Western blot detection using anti-HSV-1 glycoprotein D antibody (EMD Millipore Corp). In a separate experiment, the final CHO clone and/or V27 cells were infected with different MOIs of rHSV-AAV9 using the same approach described above. Infected cells were then incubated in 6-well plates containing an in-house medium (for CHO) or DMEM-2% FBS for V27 for two days at 33° C., in 5% CO₂ humidified incubator. Clarified viruses from lysed cultures were harvested and titrated on V27 cells by plaque assay (Kang et al., 2009. Gene Therapy 16, 229-239).

Production of rHSV-1 in BHK-21-ICP27 Pools

Recovered BHK-ICP27 pools were tested for rHSV-1 production. In brief, cells were infected with rHSV-AAV6.2 vectors (MOI=0.15 PFU) by direct inoculation in Xell HEK TF medium supplemented with either 4% FBS or under serum-free conditions. Infected cells were incubated at 37° C., 120 rpm, 5% CO₂ humidified incubator for 3-4 days. The cell viability and density were measured every day post-infection using a Vi-Cell. After 3-4 days, infected cultures were subjected to three cycles of freeze and thaw in an isopropanol dry ice bath followed by centrifugation at 4500 rpm for 15 minutes at 4° C. After centrifugation, supernatant was subjected to ultracentrifugation at 10,000 rpm for 75 minutes at 4° C. using a JA 20 rotor. The resuspended viruses were titrated on V27 cells using plaque assay according to (Kang et al., 2009).

### Statistical Analysis

One-way ANOVA with Tukey-Kramer post-hoc was used to compare GFP expression post rHSV-1 infection among CHO cell pools and to compare rAAV9-GFP titers produced from different cell pools. Two-way ANOVA was used to compare rAAV6.2-GFP titers produced from different clones at two different time points. Two-tailed Mann-Whitney test was used to compare infectivity titers of rAAV6.2-GFP and rAAV9-GFP produced in CHO or rAAV6.2-GFP and rAAV9-ZsGreen by triple transient transfection. Kruskal-Wallis test with Dunn's test for correction was used to compare rAAV6.2-GFP, rAAV9-GFP and/or rAAV9-ZsGreen titers among harvested mice tissues. GraphPad Prism version 9.1.2 (GraphPad Software Inc) was used for all tests and p-value of ≤0.05 was considered a significant difference.

### Results

### Generation of Stable CHO Pools

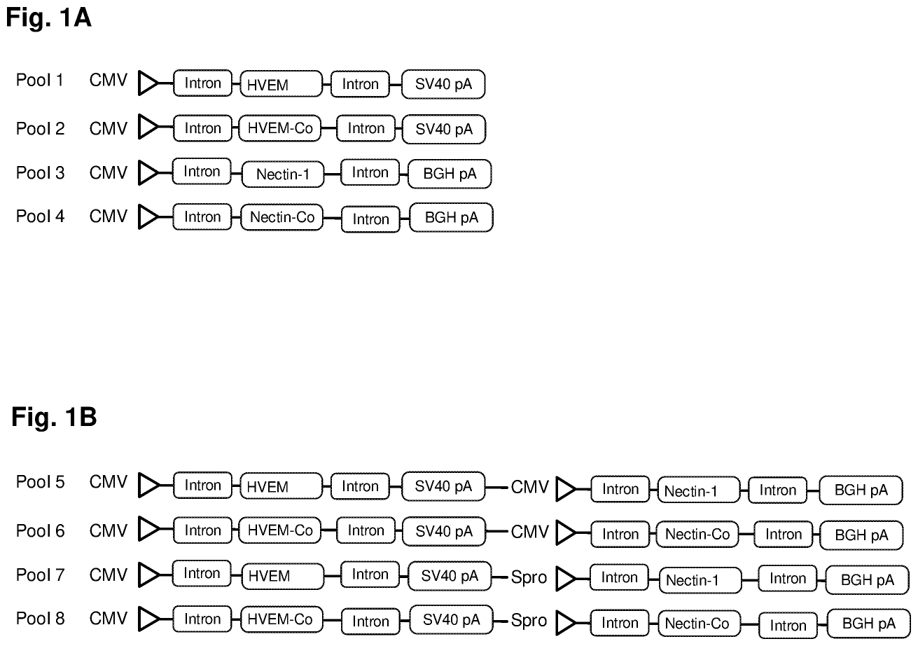

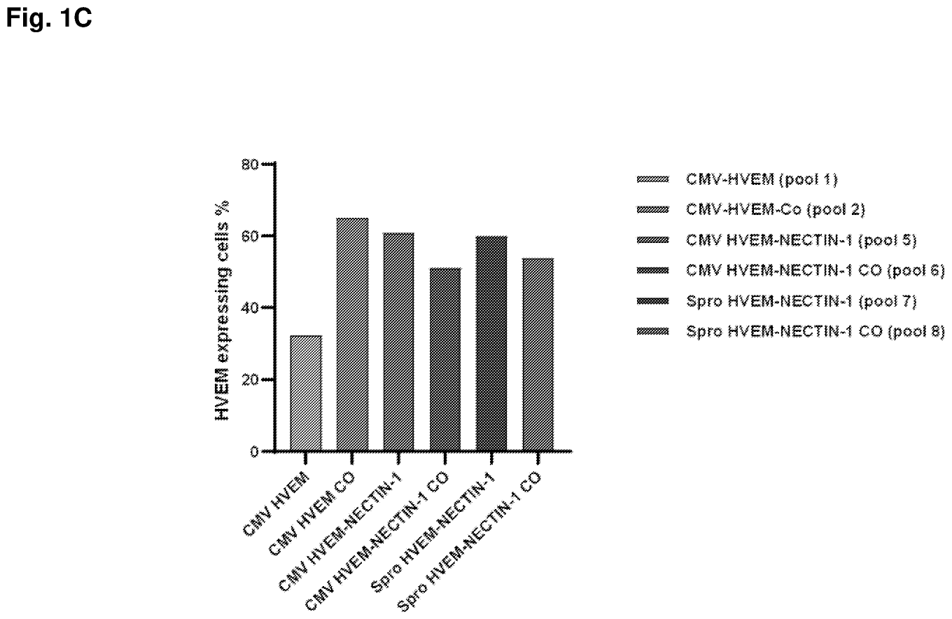

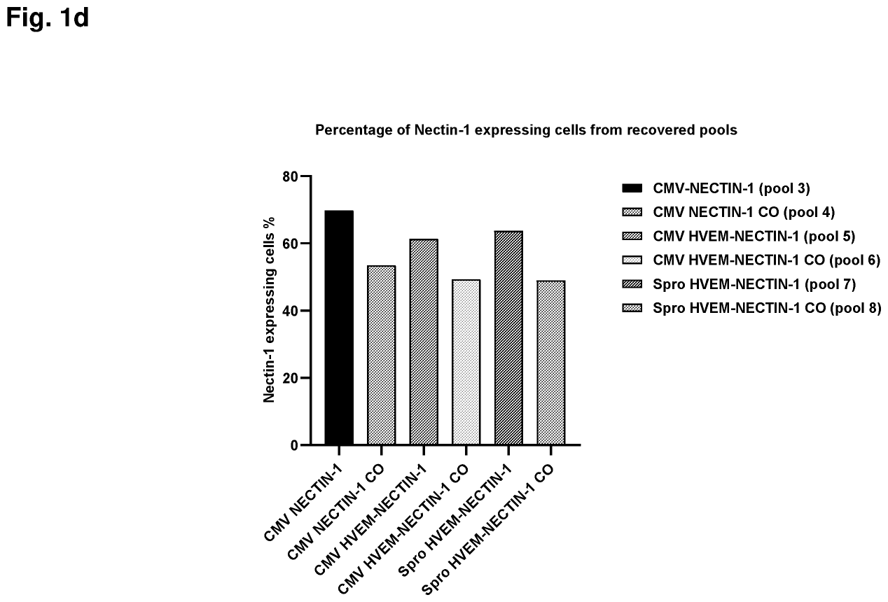

Eight different vectors using an in-house plasmid backbone were constructed successfully: (1) two vectors encoding either codon-optimized or non-codon-optimized HVEM ORF flanked by the CMV promoter and the SV40 poly A; (2) two vectors encoding either codon-optimized or non-codon-optimized Nectin-1 ORF flanked by CMV promoter and BGH poly A; (3) two vectors encoding either codon-optimized or non-codon-optimized HVEM and Nectin-1 ORFs flanked by CMV promoter, SV40 poly A and BGH poly A, respectively; and (4) two vectors encoding either codon-optimized or non-codon-optimized HVEM and Nectin-1 ORFs flanked by synthetic promoter (Spro), SV40 poly A and BGH poly A, respectively (FIGS. 1a and b). After ten days of MSX selection, an aliquot of each recovered stable cell pool (1×106 viable cells/pool) was tested for either HVEM and/or Nectin-1 receptor expression using FACS staining. Pools 1 (CMV-HVEM), 2 (CMV-HVEM-Co), 5 (CMV-HVEM-Nectin-1), 6 (CMV-HVEM-Co-Nectin-1-Co), 7 (Spro-HVEM-Nectin-1) and 8 (Spro-HVEM-Co-Nectin-1-Co) showed 32.35%, 65%, 60.9%, 51%, 60%, 53.8% HVEM expressing cells, respectively (FIG. 1c), whereas pools 3 (CMV-Nectin-1), 4 (CMV-Nectin-1-Co), 5 (CMV-HVEM-Nectin-1), 6 (CMV-HVEM-Co-Nectin-1-Co), 7 (Spro-HVEM-Nectin-1) and 8 (Spro-HVEM-Co-Nectin-1-Co) showed 69.75%, 53.45%, 61.3%, 49.3%, 63.7% and 53.8% Nectin-1 expressing cells, respectively (FIG. 1d). Interestingly, dual pools such as pools 5, 6, 7 and 8 showed good expression of HVEM and Nectin-1, indicating CMV and synthetic promoters were comparable in driving high level of expression of HVEM and Nectin-1 proteins. Moreover, no significant differences in HVEM and Nectin-1 receptor expression using either non-codon-optimized and/or codon-optimized protein versions were observed.

rHSV-1 Infection and rAAV9-GFP Production in Stable CHO Pools

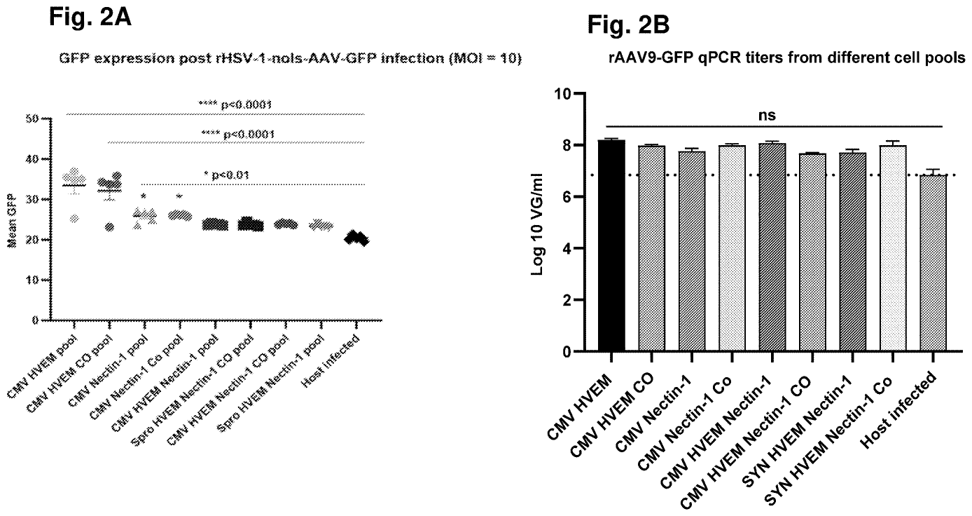

With these receptors stably expressed in these CHO pools, it is critical to understand which construct provides the best rHSV-1 infection and subsequent rAAV expression after co-infection with rHSV-1 vectors. Each of the stable pools generated above were infected with rHSV-GFP at an MOI=10, with the host CHO cells infected as a negative control. GFP expression data were collected from all the infected stable cell pools (n=8) every 12 hours post-infection to a total of 60 hours. All eight infected cell pools showed significant mean GFP expression starting at 12 hours post-infection (hpi), compared to the infected host CHO cells that showed the lowest GFP expression (FIG. 2a). The highest level of GFP expression was observed at 12 hpi in the HVEM-only expressing pools, where pool 1 and pool 2 showed the highest GFP expression (with ap-value <0.0001 compared to the infected host CHO cells) followed by pools 4 and 3 (codon optimized with ap-value <0.01 compared to the infected host CHO cells). In addition, stable cell pools 1 and 2 showed the highest GFP expression overall, with pool 1 outperforming pool 2 in the mean GFP expression overall.

One day post co-infection, cell supernatant was collected and rAAV9-GFP vector was titrated using qPCR. Pool 1 showed the highest rAAV9-GFP physical titer with average 8.2 log₁₀ vg/mL, compared to other pools number; 2, 3, 4, 5, 6, 7 and 8 that showed average 7.97, 7.76, 7.99, 8.07, 7.68, 7.71, and 7.99 log₁₀ vg/mL, respectively. However, the difference in qPCR titer for rAAV9-GFP vector produced in pool 1, compared to titers produced in other pools was not significant. The produced rAAV9-GFP titers from all pools were significant, compared to rAAV9-GFP titer obtained from infected host CHO cells CHO cells (6.84 log₁₀ vg/mL) with p<0.0001 (FIG. 2b). These data indicate that pool 1 showed the highest mean GFP expression post rHSV-GFP vector infection and the highest rAAV9-GFP titer, so this pool was selected for single cell cloning.

Generation and Testing CHO-HVEM Expressing Clones for rHSV-GFP Infection

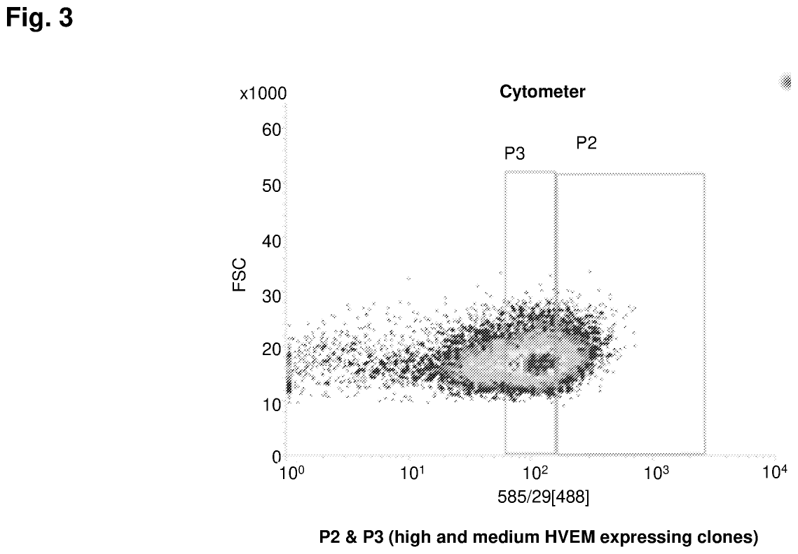

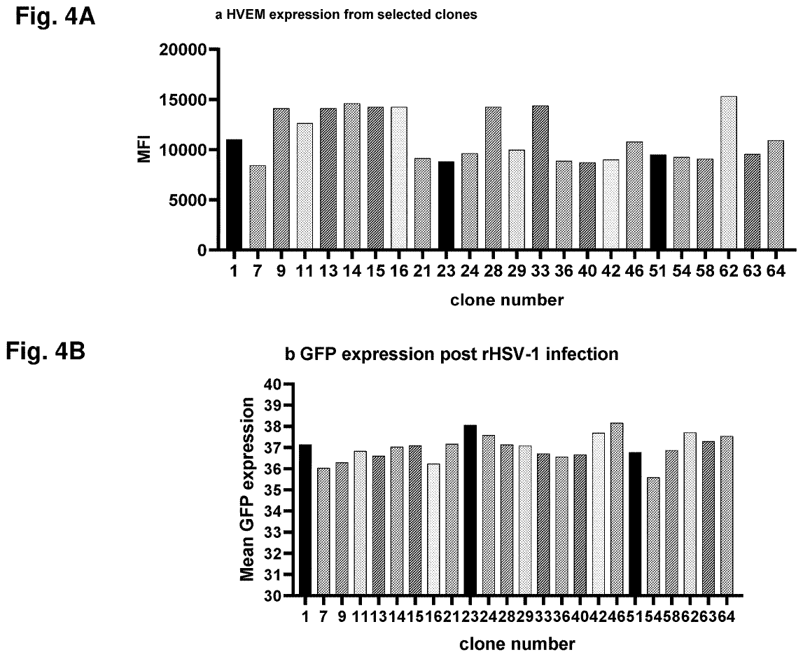

Single high and medium HVEM-expressing CHO clones (FIG. 3) were selected and deposited into two 384-well plates for two weeks using an in-house conditioned medium. Deposition of single cell/well was verified by imaging using Cellavista (Evans et al, 2015). Sixty-four clones were recovered after two weeks that showed high viability (90-95%) and good growth profiles. The selected clones were further passaged three times in 96-deep well plates contained an in-house medium supplemented with MSX. After three passages, twenty-four out of the initial 64 clones showed good HVEM expression by FACS staining (FIG. 4a). These clones were expanded and further tested for rHSV1-GFP vector infection. The mean GFP expression was calculated from two time points (24 and 48) hpi. No statistically significant difference in the mean GPF expression levels was detected among the tested clones (FIG. 4b).

rAAVs Production in CHO-HVEM Expressing Clones

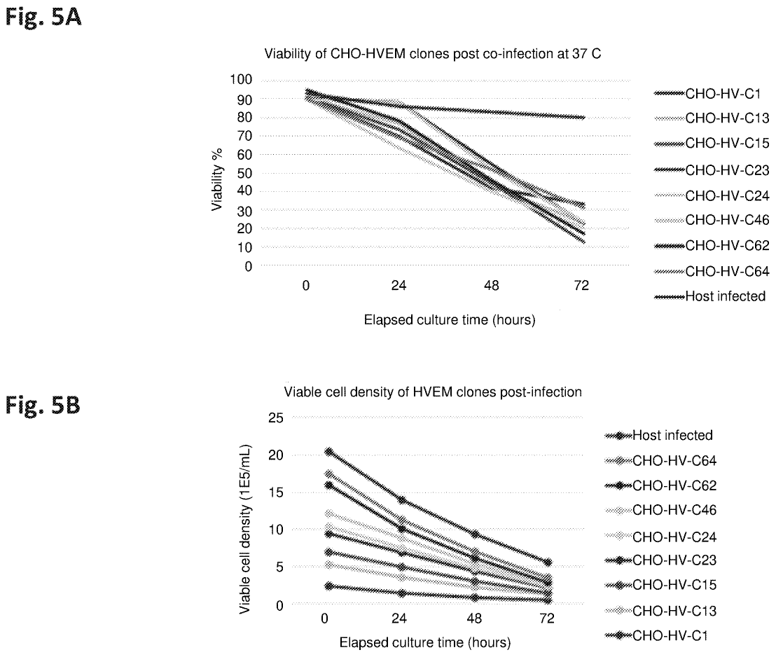

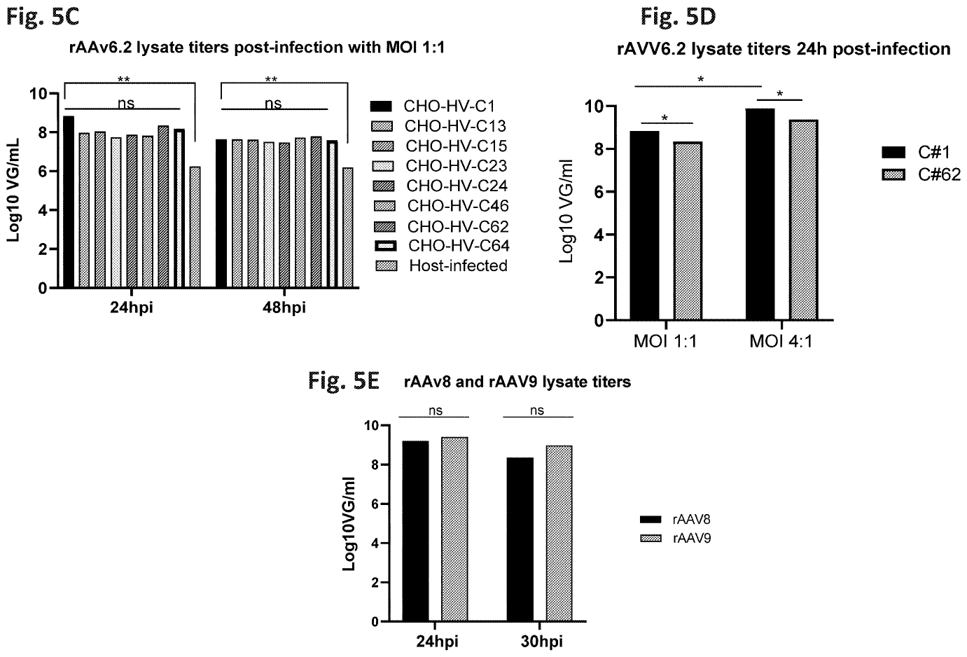

Eight clones (called CHO-HV-C1, CHO-HV-C13, CHO-HV-C15, CHO-HV-C23, CHO-HV-C24, C CHO-HV-C46, CHO-HV-C62 and CHO-HV-C64) that showed highest mean GFP expression post rHSV-GFP vector infection were selected to assess their capability to produce rAAV via the co-infection with two rHSV-1 vectors, one containing the AAV2 rep and AAV6.2 cap genes and the other containing the GFP gene at an MOI 1:1. Post infection, the viability of the eight clones dropped rapidly over the days, compared to only a slight drop (3-7%) of the infected host CHO cells over the course of the co-infection (FIG. 5a) when incubated at 37° C. Accordingly, the viable cell density (VCD) of all co-infected clones also showed a significant drop compared to the minor drop (0.3×10⁶/mL) of the infected host CHO cells (FIG. 5b). It seems that AAV rep protein exerts a deleterious effect on the metabolism of the infected engineered CHO cells as indicated by the sharp drop in the cell viability and the VCD compared to the host CHO cells in which rHSV-1 vectors undergo degradation after cellular entry when co-infected cells incubated at 37° C. Therefore, we thought to minimize this deleterious effect by lowering incubation temperature.

Interestingly, clone #1 (called CHO-HV-C1) produced the highest rAAV6.2-GFP vector titer per 1 mL cell lysate (~8.83 log₁₀ vg/mL; from 1×10⁶ cells) at 24 hpi, compared to other clones that produced between 7.74 and 8.34 log₁₀ vg/mL. However, the difference in rAAV6.2-GFP titers produced in CHO-HV-C1 clone was not significant to those produced by other tested clones (p=0.89). However, they were all significantly higher than the co-infected host CHO cells that produced 6.24 log₁₀ vg/mL 24 hpi. (FIG. 5c). rAAV6.2-GFP titers from all eight co-infected clones slightly dropped at 48 hpi (FIG. 5c).

Clones CHO-HV-C1 and CHO-HV-C62 produced the highest titers of rAAV6.2-GFP vector. Clone CHO-HV-C1 is a high HVEM expressing clone, whereas CHO-HV-C62 clone is a medium HVEM expressing clone. In a subsequent experiment, final selected clones (CHO-HV-C1 and CHO-HV-C62) were tested for rAAV6.2-GFP vector production using MOIs of 2:1, 3:1, and 4:1 of rHSV-AAV6.2 and rHSV-GFP, respectively.

Interestingly, MOIs 2:1 and 3:1 did not show significant improvement in rAAV6.2-GFP titers per 10⁶ cells lysate at 24 hpi, compared to that obtained using MOI 1:1 (data not shown). On the other hand, MOI 4:1 significantly improved rAAV6.2-GFP physical titers produced in CHO-HV-C1 and CHO-HV-C62 clones (p=0.0211). In addition, CHO-HV-C1 clone outperformed CHO-HV-C62 for production of rAAV6.2-GFP vector at MOI 4:1 and produced, yielding 9.89 and 9.37 log₁₀ vg/mL, respectively per 10⁶ cells lysate at 24 hpi (p=0.0261) (FIG. 5d), indicating that MOI of rHSV-1 has a major impact for the rAAV6.2-GFP in CHO cells.

As CHO-HV-C1 was the most productive clone for rAAV6.2-GFP production, further testing on its capacity for the production of other AAV serotypes 8 and 9 expressing the GFP transgene was tested. Using the same optimal infection parameters described in the previous experiment, average titers of 9.21 and 9.4 log₁₀ vg/mL per 1×10⁶ cells cell lysate for rAAV8-GFP and rAAV9-GFP vectors, respectively, were produced at 24 hpi. Harvesting at 30 hpi showed decreased titers to 8.36 and 8.98 log₁₀ vg/mL for rAAV8-GFP and rAAV9-GFP vectors, respectively (FIG. 5e). These data indicate that the infection parameters described above work across tested serotypes. Additionally, cell lysate produced the highest physical titers of the AAV tested serotypes at 24 hpi compared to titers at 30 and 48 hpi.

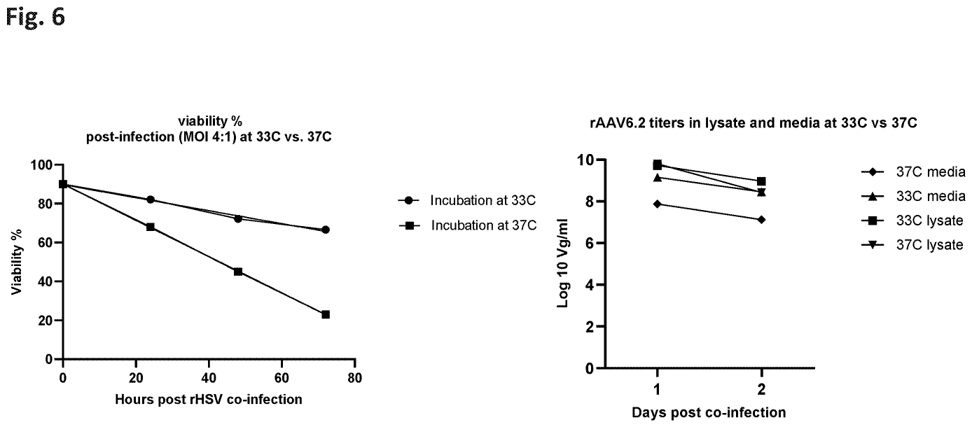

Cell viability of the co-infected CHO-HV-C1 cells for production of either rAAV8-GFP and/or rAAV9-GFP vectors sharply dropped 24 hpi similar to the case in rAAV6.2-GFP vector production, which indicates that cell viability drop is not an AAV serotype-specific. A sharp decline in the cell viability after rHSV-1 vector co-infection may affect the final rAAVs titers, so the effect of the temperature shift from 37° C. to 33° C. was tested, which showed a significant improvement for both cell viability and rAAV6.2-GFP titer post co-infection (FIG. 6). Therefore, co-infection with MOI 4:1 (rHSV-1 AAV6.2:rHSV-1-GFP) at 33° C. was tested. Only a slight drop (5-7%) in cell viability for co-infected cultures with a slight improvement in rAAV6.2-GFP titers from lysate and ~two-fold titer increase in the medium at 24 hpi compared to that incubated at 37° C. was seen (data not shown). This finding indicates that incubation temperature post co-infection is an essential factor for either cell viability and/or rAAV production in the CHO platform.

Analytical Characterization of CHO-Derived rAAVs

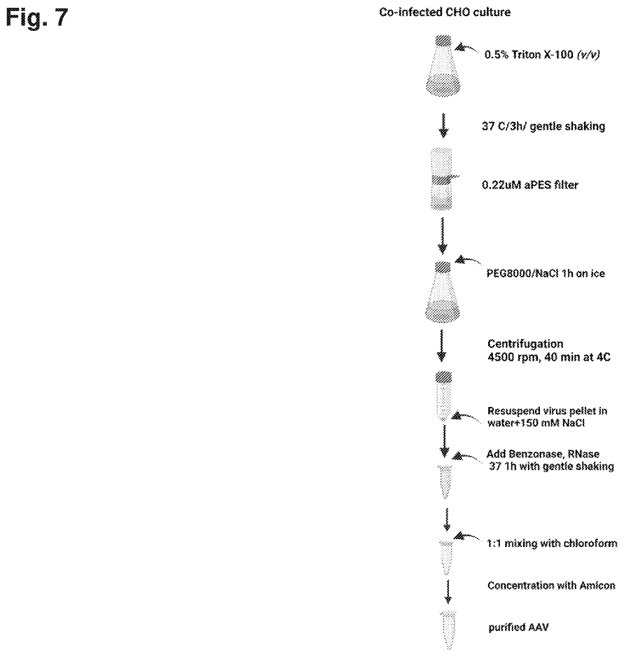

The whole process of harvesting and purifying the rAAV vectors takes one day to perform in the in-house developed PEG-chloroform method (FIG. 7). In brief, the entire infected culture is lysed for 1-3 hours under gentle shaking with 0.5% Triton X-100 (v/v). The Triton-treated culture is then centrifuged for 5 minutes at 1200 rpm, and the supernatant is then 0.2 μm-filtered using a PES filter. The filtrate is mixed with 14 volume of 40% PEG8000/5 M NaCl for 1 hour on ice followed by centrifugation at 4500 rpm for 40 minutes at 4° C. The PEG virus pellet is resuspended in resuspension buffer and subjected to Benzonase (50 U/ml), RNase A (20 μg/ml) for 1 hour at 37° C. with tube mixing every 15 minutes. After Benzonase treatment, the mixture is mixed with chloroform at ratio 1:1 and centrifuged for 5 minutes at 12,000 rpm. After centrifugation, chloroform is evaporated under biological safety cabinet and the aqueous layer is collected and then concentrated. The final purified rAAV is stored at −80° C.

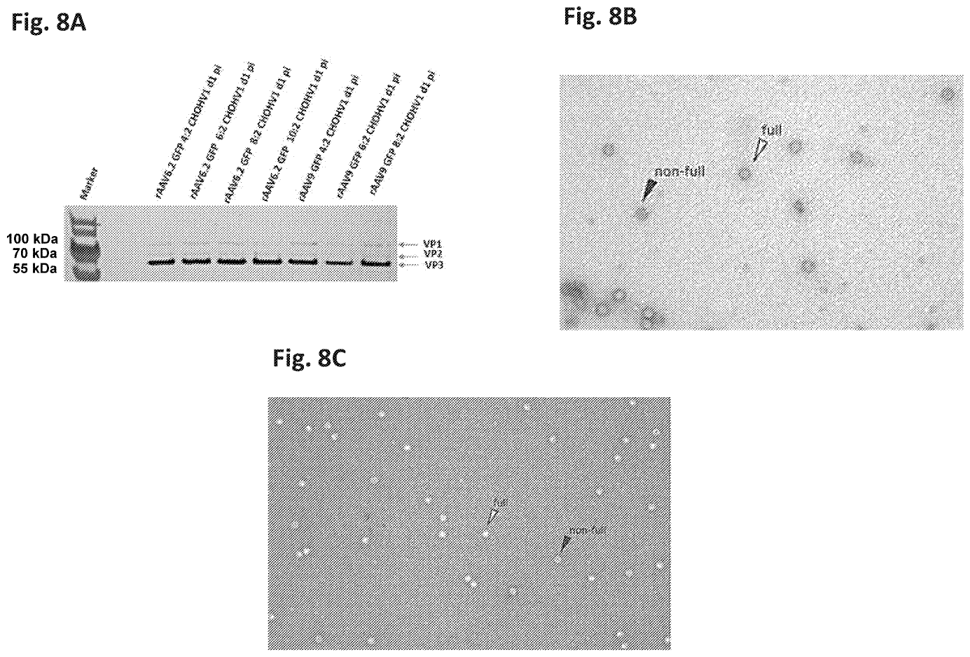

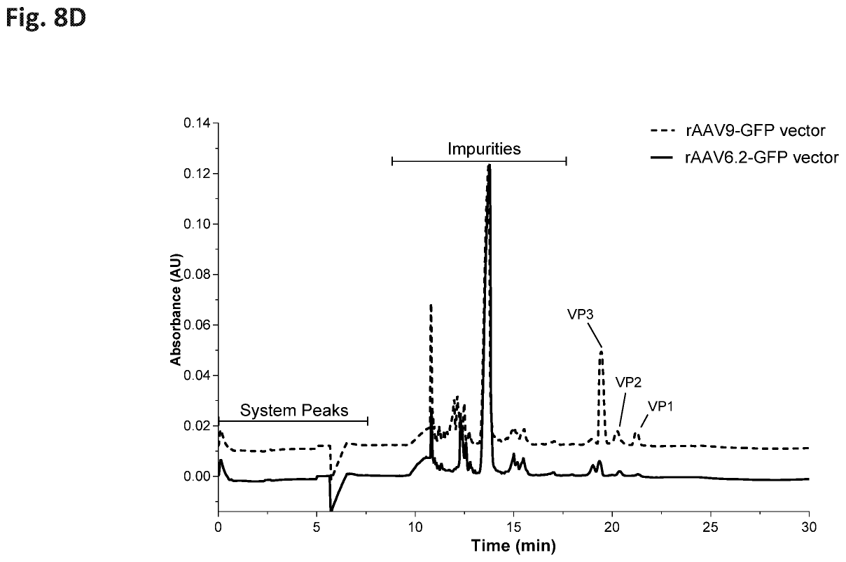

Western blotting of equal volumes of the purified rAAVs produced in the CHO-HV-C1 clone using different MOIs of HSV-1s showed comparable expression of VP1, VP2, and VP3 capsid proteins (FIG. 8a). Moreover, examination of the purified rAAV6.2-GFP and rAAV9-GFP vectors using the mini-TEM showed 91 and 79.5% full capsids for rAAV6.2-GFP and rAAV9-GFP, respectively (FIGS. 8b and 8c). Additionally, analysis of the VP1:VP2:VP3 molar ratio of these rAAVs were consistent with those reported in the literature (FIG. 8d). These data show that rAAV vectors produced in the CHO cells have good expression of capsid proteins with a high percentage of AAV full capsids. However, the high percentage of full capsids obtained may be related to the purification method used, so it is worth reinvestigating the full capsid percentage after using other purification methods such as chromatographic method.

Residual Infectious HSV-1 from CHO-Derived rAAVs

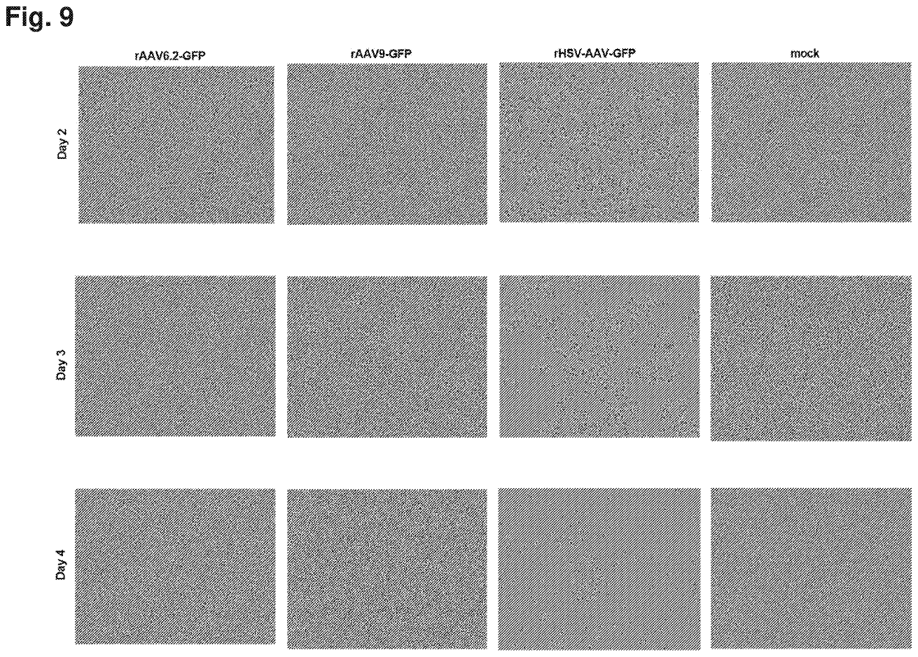

Purified rAAV6.2-GFP and rAAV9-GFP vectors were examined for any residual infectious HSV-1 vectors in the purified drug substance by inoculating 10 log₁₀ vg/mL of each vector on an HSV-1-complementing cell line (V27), with rHSV-GFP vector (MOI 0.15 PFU/cell) as a positive control. No cytopathic effect appeared in wells inoculated with either purified rAAV6.2-GFP or rAAV9-GFP vectors 4 days post-infection, whereas a typical cytopathic effect, in form of rounding of infected cells and cell sheet detachment, appeared in the well inoculated with rHSV-GFP vector starting on day 2 post-infection, resulting in complete detachment of the cell sheet on day 3 post-infection (FIG. 9). These data indicate that the in-house developed purification method is highly efficient in inactivating rHSV-1 vectors, and no residual infectious rHSV-1 was detected in the purified rAAV.

Infectivity and In Vitro Transduction of CHO-Derived rAAVs

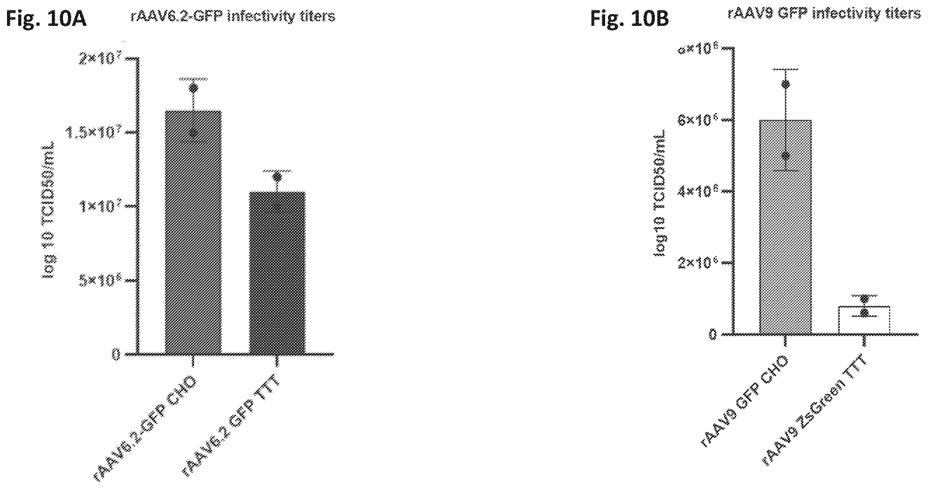

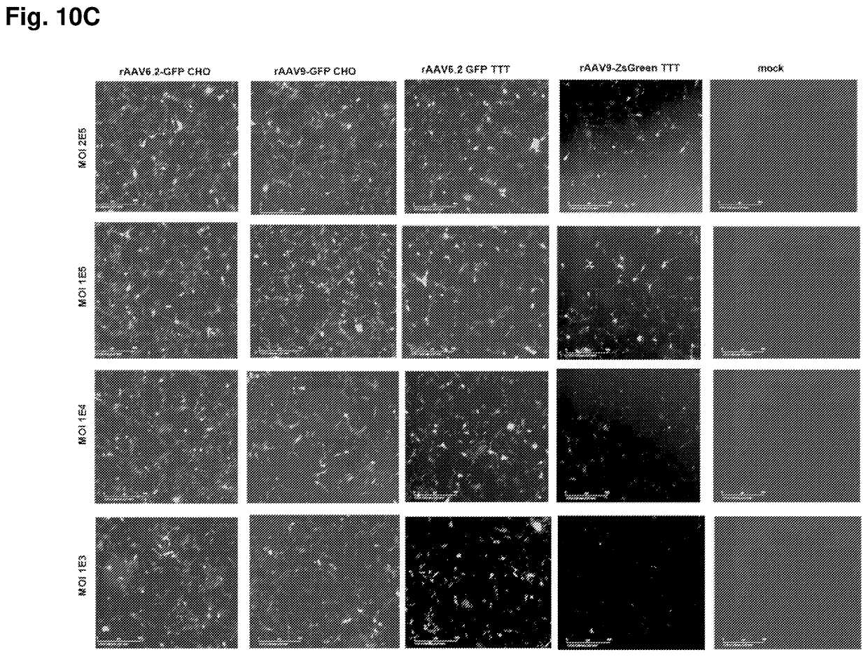

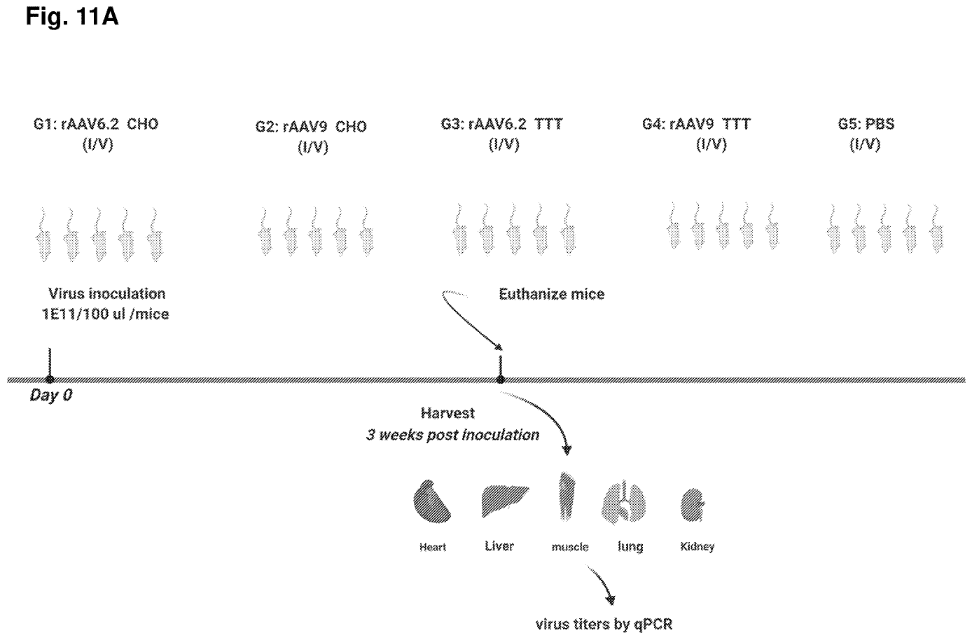

The infectivity and the in vitro transduction potency of rAAVs produced in CHO cells and purified using PEG-chloroform was tested to compare them to those produced using the standard triple transient transfection method and purified with the affinity-based chromatography. For infectivity, the GFP expression from infected wells was recorded on day 5 post-infection using the IncuCyte default settings. The rAAV6.2-GFP vectors produced in the CHO-HV-C1 clone (called rAAV6.2-CHO) and the rAAV6.2-GFP produced in HEK293 cells (called rAAV6.2-GFP TTT) showed comparable infectivity titers of 1.65×10⁷ and 1.1×10⁷ tissue culture infective dose 50 (TCID₅₀/mL), respectively (FIG. 10a). On the other hand, rAAV9-GFP vectors produced in CHO-HV-C1 clone (called rAAV9-GFP CHO) showed 6×10⁶ TCID₅₀/mL, compared to rAAV9-ZsGreen vectors produced in HEK293 cells using triple transient transfection called (rAAV9-Zs-Green-TTT) that showed 6×10⁵ TCID₅₀/mL (FIG. 10b).

Different multiplicity of transfection (MOT), ranging from 2×10⁵ down to 1×10³ vg/cell from the aforementioned four vector preparations, were tested in Ad293 cells for comparison of transduction, and the mean GFP expression was recorded on day 3 post-infection using the IncuCyte default settings. rAAV6.2-GFP-CHO and rAAV6.2-GFP-TTT showed potent transduction at all the MOT tested. On the other hand, rAAV9-GFP-CHO showed higher transduction efficacy compared to rAAV9-ZsGreen-TTT (FIG. 10c), consistent with the observed infectivity data. These data indicate that rAAV6.2-GFP-CHO and rAAV9-GFP-CHO have good an in vitro infectivity and transduction activity. Additionally, the higher infectivity and transduction of rAAV9-GFP-CHO compared to rAAV9-Zs-Green-TTT observed here may relate to the difference in the percentage of the full capsids, purification methods and the formulation buffers used for each of the different sample preparations.

### Biodistribution of CHO-Derived AAVs

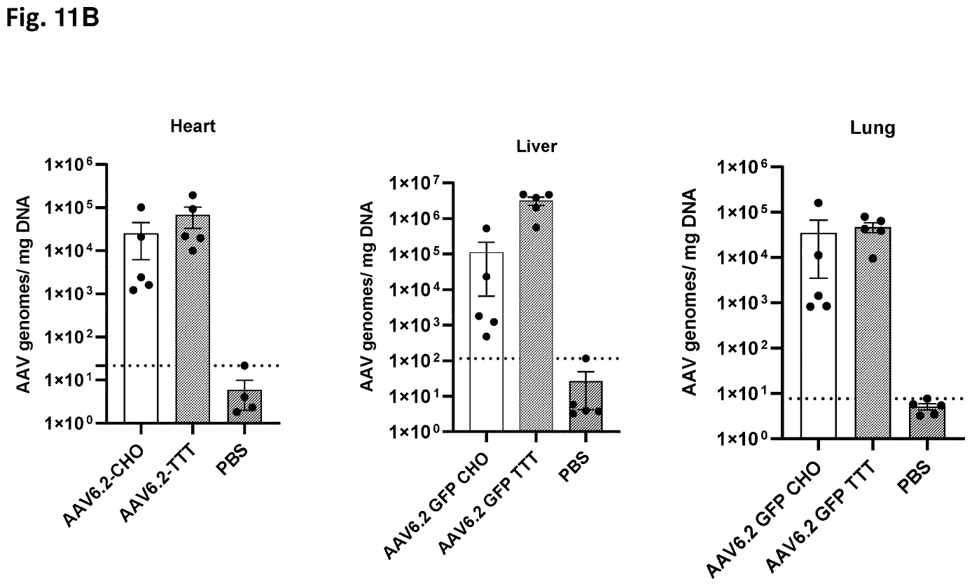

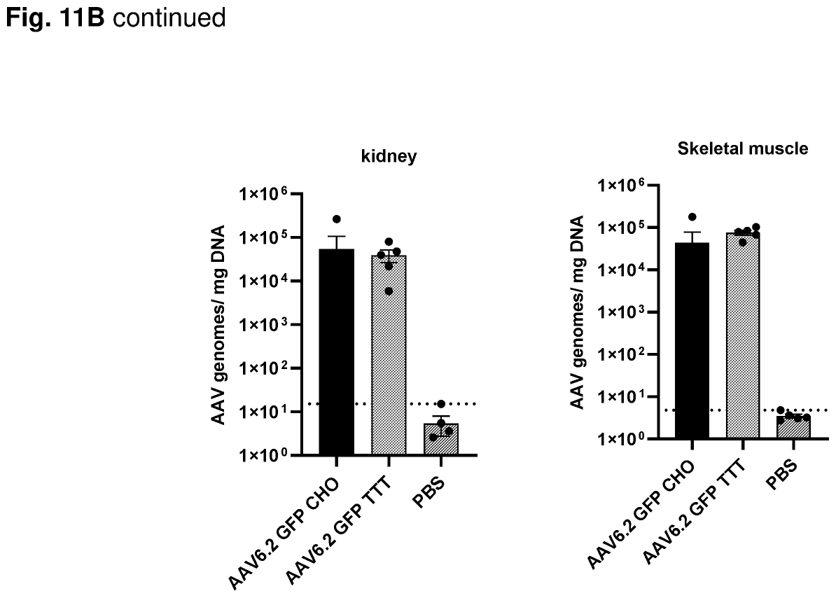

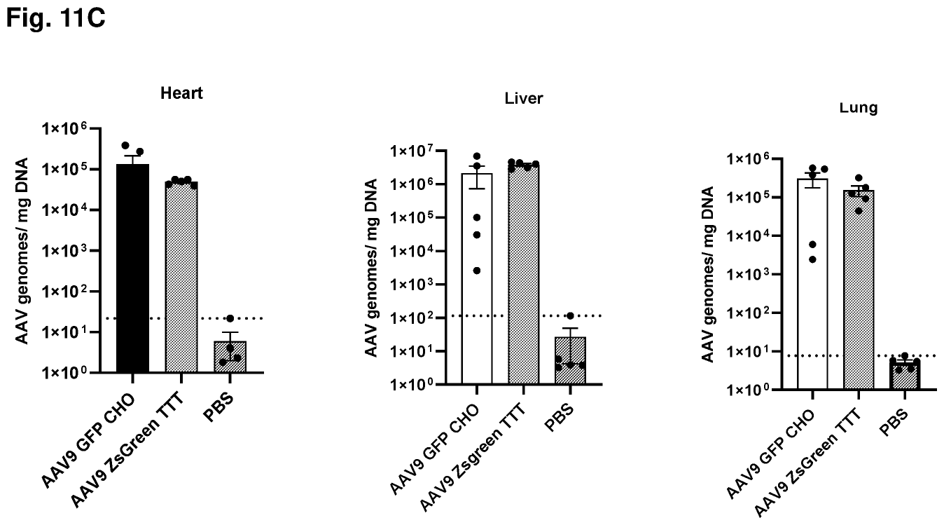

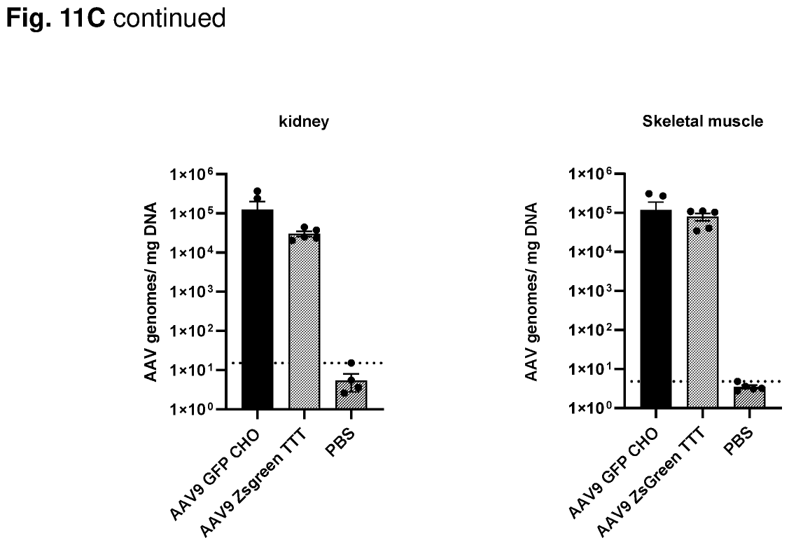

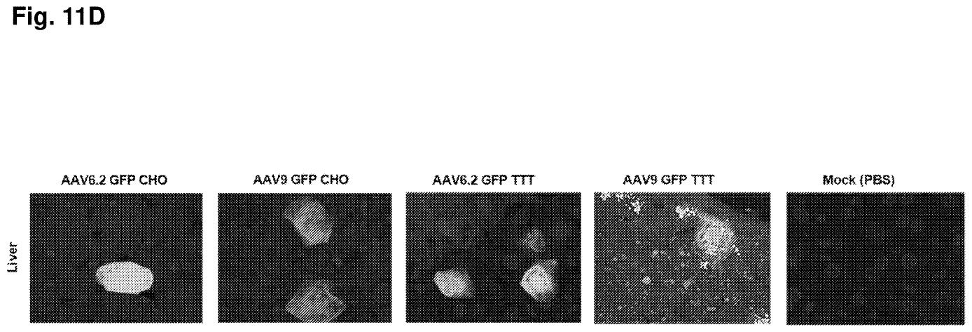

The biodistribution of rAAV6.2-GFP-CHO and rAAV9-GFP-CHO in parallel with the rAAV6.2-GFP-TTT and rAAV9-Zs-Green-TTT was assessed to determine if the in vivo behavior mimicked the in vitro data. Twenty five three-week old mice were divided into five groups (n=5 per group). The mice were inoculated with either rAAV6.2-GFP-CHO (G1), rAAV9-GFP-CHO (G2), rAAV6.2-GFP-TTT (G3), rAAV9-Zs-Green-TTT (G4), or PBS (G5). All mice were inoculated with either 10¹¹ vg rAAV or 100 μL of PBS intravenously in the tail vein, as per their grouping. Three weeks post-inoculation, the inoculated mice were euthanized, and the tissues (heart, liver, lung, kidney, and skeletal muscle), which have high tropism for these tissues, were harvested from each inoculated animal (FIG. 11a). These tissues were assessed for rAAV titer in homogenized tissue using qPCR (targeting GFP and/or Zs-Green gene) and histopathologic examination using confocal microscopy. For qPCR, mice from G1 showed lower GFP copy numbers than mice from G3 in all harvested tissues except for the kidneys, where mice from G1 showed higher mean GFP copies/mg~5.38×10⁴ AAV genomes/mg DNA compared to 3.89×10⁴ AAV genomes/mg DNA (FIG. 11b), although these differences are not statistically significant. Additionally, rAAV6.2-GFP copy numbers from livers from G1 and G3 showed the highest GFP copies in all inoculated mice, compared to titers from other tissues from both groups. Interestingly, mice in G2 showed higher mean GFP copies/mg DNA in heart, lung, kidney, and skeletal muscle than those from G4. However, livers from G4 showed higher mean GFP copies/mg DNA than livers from G2 with mean titers of 3.79×10⁶ and 2.11×10⁶ vg/mg DNA, respectively. Moreover, GFP titers from livers from either G2 and/or G4 showed the highest GFP copies in all inoculated mice from both groups, compared to titers from other tissues (FIG. 11c).

Thin liver tissue sections from the five groups were prepared and examined for the GFP expression using confocal microscopy. Clear GFP and/or Zs-Green signals were observed in livers of all inoculated groups except G5 (FIG. 11d), as expected from the qPCR data. Additionally, quantification of GFP and Zs-Green from liver slides using a slide scanner with an in-house developed script showed that liver sections from G3 showed significant biodistribution compared to those from G1 that correlates with the qPCR data. Additionally, liver sections from G4 also showed higher Zs-Green signals than GFP signals from G2; however, the difference was not significant. No GFP signal was detected from the mock infection group (G5) inoculated with sterile PBS. These data indicate that rAAV produced in CHO cells show good in vivo transduction after tail vein injection. Additionally, lower GFP in liver sections, particularly from G1, may relate to several factors such as presence of impurities that cannot be completely eliminated with PEG-Chloroform method.

Engineering CHO-HV-C1 Cells for rHSV-1 Production

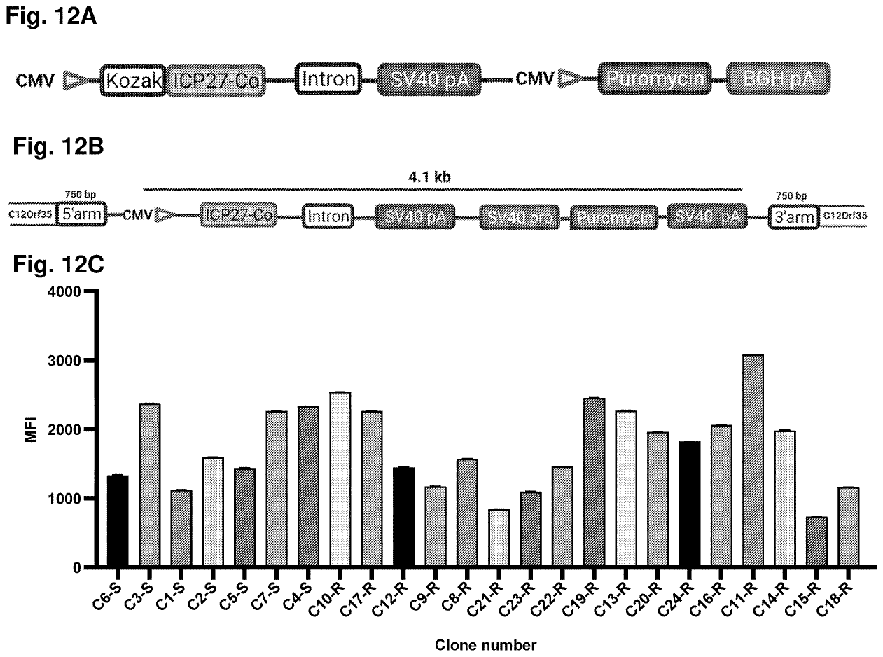

For random-integration, an in-house constructed linearized plasmid contained synthesized Chinese hamster codon-optimized HSV-1 ICP27 ORF downstream the CMV promoter and upstream SV40 poly A and puromycin ORF downstream the CMV promoter and BGH poly A was used for transfection (FIG. 12a).

For site-integration, two plasmids were constructed, the first contained codon-optimized ICP27 ORF downstream the CMV promoter and upstream SV40 poly A followed by puromycin cassettes that are flanked by the SV40 promoter and SV40 poly A. The total length of the two cassettes was 4.1 kb flanked by the right and left homology arms (750 bp each) (FIG. 12b). The second contained synthetic sgRNA for CHO exon 1 C12orf35. Pools from either random and/or site-integration were recovered after 3 weeks of double selection using 5 μg/mL of puromycin and 50 μM MSX/mL. After three passages in an in-house medium with double selection, twenty-four clones, including seven site-edited clones (clone 1-7) and 17 random-integrated clones showed high growth viability and ICP27 expression (FIG. 12c) were selected. Random integrated clone #11 (called CHO-HV-ICP27-C11) that showed the highest growth profile and ICP27 expression was selected for further testing against rHSV-1 vectors production.

Infection and Production of rHSV-1 Vectors in CHO-HV-ICP27-C11 Cells

The CHO-HV-ICP27-C11 clone was infected with rHSV-AAV9 (MOI=10) and incubated at 37° C. for 2 hours for virus adsorption. After 2 hours, infected cells were washed twice with sterile 1×PBS to remove any virus residues. The infected cells were then incubated for 24 hours at 37° C. in a 5% CO₂ static humidified incubator. On second day, 1 mL of clarified supernatant from infected cells was passaged on V27 cells. Appearance of cell rounding and detachment of the infected cell sheet appeared two days post infection. Additionally, expression of HSV-1 glycoprotein D (gD) was observed in the V27 cell lysate after infection with the rHSV-1 virus propagated in CHO-HV-ICP27-C11 clone (data not shown). This result indicates that CHO-HV-ICP27-C11 clone supports productive infection for rHSV-1 vectors.

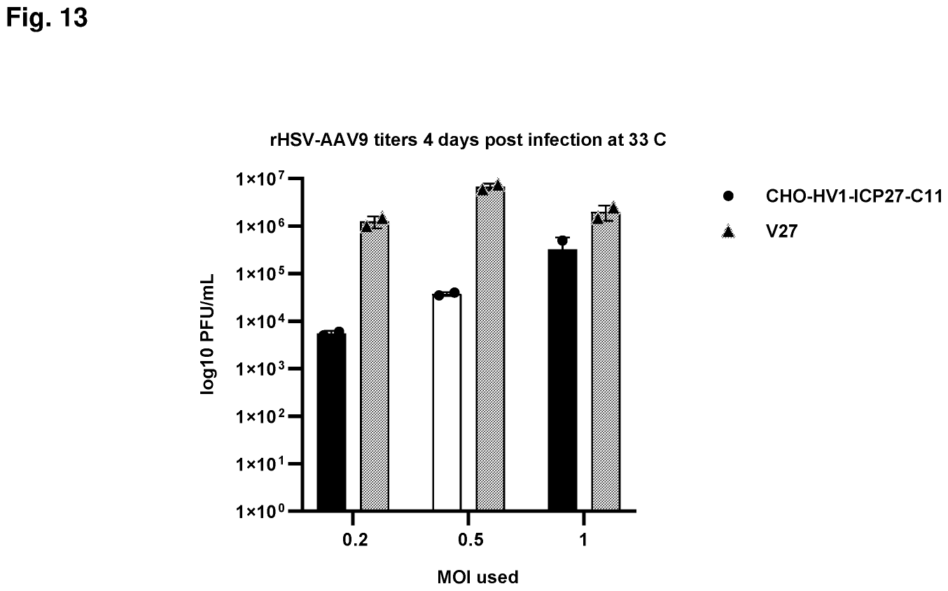

Therefore, the production capability of CHO-HV-ICP27-C11 clone to the V27 cells was compared. Different MOIs (0.2, 0.5 and 1 PFU/cell) of rHSV-AAV9 were used to infect either CHO-HV-ICP27-C11 or V27 cells using serum-free in-house medium or DMEM supplemented with 2% (v/v) FBS, respectively. The infected cell cultures were incubated for 4-5 days at 33° C., 5% CO₂ in a humidified incubator. rHSV-AAV9 from infected cell cultures were released by three freeze-thaw cycles and titrated by plaque assay on V27 cells. CHO-HV-ICP27-C11 cells produced significantly lower rHSV-AAV9 titers than V27 cells in which CHO-HV-ICP27-C11 clone produced 5×10³, 4×10⁴, and 3.2×10⁵ PFU/mL, compared to V27 cells that produced 1×10⁶, 6.75×10⁶ and 2×10⁶ PFU/mL, respectively, at MOIs of 0.2, 0.5 and 1 PFU/cell on day 2 post-infection (FIG. 13).

### Production of HSV-AAV6.2 Vector in BHK-21-ICP27 Pools

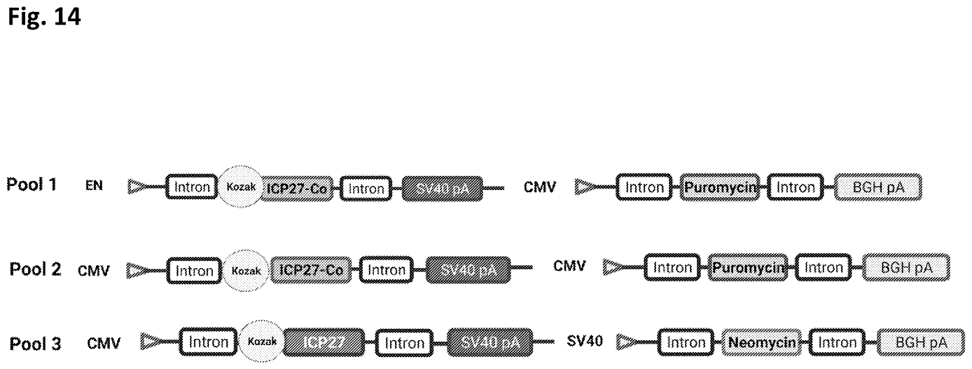

Three recovered BHK21-ICP27 pools (FIG. 14) were expanded and banked. Aliquots of pools (1×10⁶ viable cells/mL) in 30 ml shake flask were tested for HSV-AAV6.2 vector production either in presence of 4% FBS or in serum-free condition. The viability percentage of the infected culture dropped slightly on day 1 post infection from 87% to 85.5% and 84.4% for cultures supplemented with 4% FBS and 0% FBS, respectively. On day 2 post-infection, cell viability dropped to 71.6% and 65.1% for cultures supplemented with 4% FBS and 0% FBS, respectively. On day 3 post-infection, cell viability significantly dropped to 45.1% and 44% for cultures supplemented with 4% FBS and 0% FBS, respectively. In addition, viable cell density dropped on average 1×10⁵ cells/ml daily post-infection for both infected cultures (data not shown).

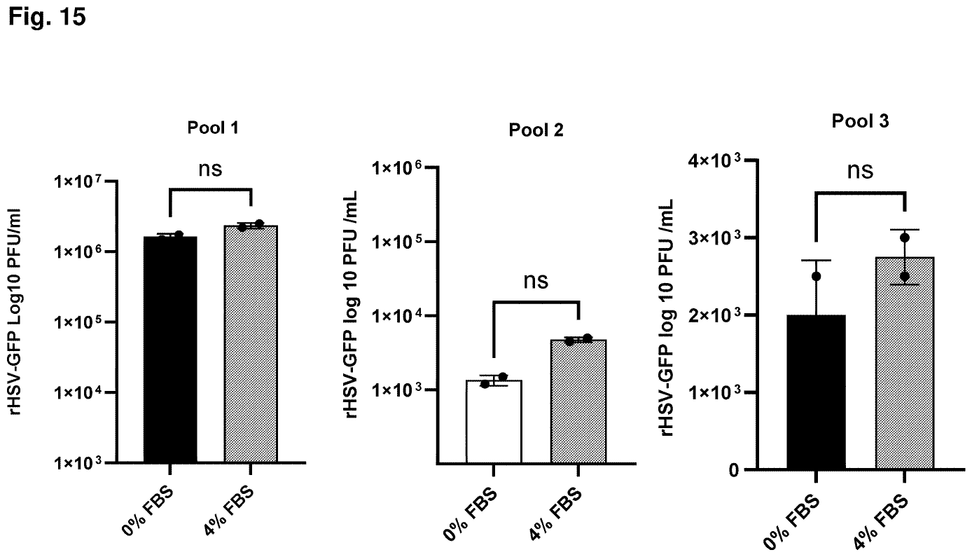

Harvested viruses were titrated on V27 cells using plaque assay. The infected BHK-21-CMV-ICP27 pool supplemented with 4% FBS produced 3.5×10⁶ PFU/ml, compared to 1.2×10⁶ PFU/ml produced by serum-free infected BHK-21-CMV-ICP27 pool (FIG. 15).

Suspension, serum-free adapted CHO cell pools expressing either HVEM and/or Nectin-1 necessary for rHSV-1 entry and infection were successfully engineered. All engineered stable CHO pools showed significant susceptibility to rHSV-1-GFP vector entry and infection as evidenced by GFP expression, compared to the wildtype CHO cells. The CMV-HVEM and CMV-HVEM-CO pools, which express non-codon-optimized and CHO codon-optimized HVEM gene, respectively, outperformed all other pools in GFP expression for five different time points post-infection with rHSV-GFP vector, compared to the wildtype-infected CHO cells. These data are in agreement with the previous publications which reported that engineered CHO express HVEM receptor became permissive to HSV-1 entry and infection (Montgomery et al, Cell Vol. 87, 427-436.).

Interestingly, pool #1 (called CMV-HVEM) also outperformed all other stable CHO cell pools in producing high rAAV9-GFP vector physical titers from cell lysate 24 hpi, which led to generation of monoclonal cells from this pool although the difference in titer was not significant to other pools. Using high-throughput methods, the top 24 CHO-HVEM expressing clones were selected that showed high; growth profile and HVEM expression. These were narrowed down to eight clones (C #1, C #13, C #15, C #23, C #24, C #62 and C #64), which showed the highest GFP expression post rHSV-nols-AAV-GFP infection for different time points. These final eight clones were further tested for rAAV6.2-GFP vector production starting with MOI 1:1 of (rHSV-AAV6.2:rHSV-nols-AAV-GFP) for three days at 37° C. in an in-house medium. There was a rapid drop in both cell viability and viable cell density starting at 24 hpi. Both cell medium and cell lysate were harvested at 24 hpi and 48 hpi points and then tested them for rAAV6.2-GFP physical titers using Q-PCR. The highest rAAV6.2-GFP physical titer was detected from cell lysate from all co-infected clones at 24 hpi, then the titer was slightly dropped at 48 hpi. Lower rAAV6.2-GFP physical titers in medium from all co-infected clones on different time points were detected (data not shown), which indicates rAAV6.2-GFP vector is mainly a cell-associated from CHO platform. Moreover, harvesting at different time points such as 30 hpi did not show improvement in the final rAAV6.2-GFP physical titer, compared to that obtained at 24 hpi. These findings are different than other systems used AAV-HSV-1 based-production, such as HEK293, in which the peak of rAAVs production was achieved at 52 h post co-infection (Kang et al., 2009). Interestingly, these tested final eight clones were variable in HVEM expression. For example, clones numbers (C #1, C #23 and C #24) were high HVEM expressing clones, whereas clones numbers (C #13, C #15, C #46, C #62 and C #64) were medium HVEM expressing clones that may have an effect on rAAVs production.

Clones C #1 (called CHO-HV-C1) and C #62 (called CHO-HV-C62) produced the highest physical titers for rAAV6.-GFP vectors 24 hpi, so different MOIs were tested on these two clones to improve the final rAAV6.2-GFP titers. MOIs (2:1, 3:1 and 4:1:6:1, 8:1 and 10:1 from rHSV-AAV6.2 and rHSV-GFP, respectively) were tested. MOIs 2:1 and 3:1 did not produce significantly improved titers, compared to MOI 1:1. Interestingly, MOI 4:1 produced ~x 10¹⁰ vg/mL (10¹³ vg/L) and ~10^{9.37} vg/mL (10^{12.37} vg/L) from clones; CHO-HV-C1 (high HVEM expressing) and CHO-HV-C62 (medium HVEM expressing), respectively, which indicates positive correlation between HVEM expression and rAAVs production. Interestingly, no significant increase in titers of rAAV6.2-GFP produced using MOIs 6:1, 8:1 and 10:1, compared to MOI 4:1 was detected (data not shown). Therefore, CHO-HV-C1 was further chosen to be tested for production of other AAV serotypes, such AAV8 and AAV9 using different MOIs, such as 1:1, 2:1, 3:1 and 4:1. The MOIs 1:1, 2:1 and 3:1 produced ~10⁸ vg/mL (10¹¹ vg/L), whereas MOI 4:1 improved the final titers to 10^{12.21} and 10^{12.40} vg/L for rAVV8-GFP and rAAV9-GFP, respectively. Interestingly, good rAAV8 and rAAV9 titers were detected from cell medium 24 hpi using MOI 4:1 (data not shown), which indicates that rAAV8-GFP and rAAV9-GFP are not cell-associated as rAAV6.2-GFP in CHO platform. Thus, for the CHO platform only low MOI is needed for production of rAAV6.2-GFP, rAAV8-GFP and rAAV9-GFP in contrast to other studies reported high MOI such as 12:2 as optimal MOI to produce rAAVs (Kang et al., 2009).

The cell viability of co-infected cells from CHO-HV-C1 for both rAAV8-GFP and rAAV9-GFP vector production sharply dropped 24 hpi similar to the case in rAAV6.2-GFP vector production, which indicates that this drop was not AAV serotype specific. It was believed that a sharp decline in cell viability after rHSV-1 vectors co-infection may affect the final rAAVs titers, so the effect of temperature shift from 37° C. to 33° C. was tested and showed significant improvement for both cell viability post co-infection and for rAAV6.2-GFP titer (data not shown). It has been reported that HSV-1 vectors stability was 2.5-fold greater at temperature 33° C. than 37° C., and HSV-1 synchronous infection incubated at 33° C. produced 2-fold higher amounts of vectors than those incubated at 37° C. (Wechuck et al., 2002). Therefore, co-infection with MOI 4:1 (rHSV-1 AAV6.2:rHSV-1-nols-AAV-GFP, respectively) was tested at 33° C., 120 rpm shaking. Interestingly, at 33° C., a slight drop (5-7%) in cell viability of co-infected cultures was found with slight improvement in rAAV6.2-GFP physical titers from lysate and ~two-fold rAAV6.2-GFP titers increase in medium produced at 24 hpi, compared to that incubated at 37° C. (data not shown). This finding indicates that shifting temperature after co-infection to 33° C. enhances the cell viability and the final rAAV6.2-GFP production from CHO platform.

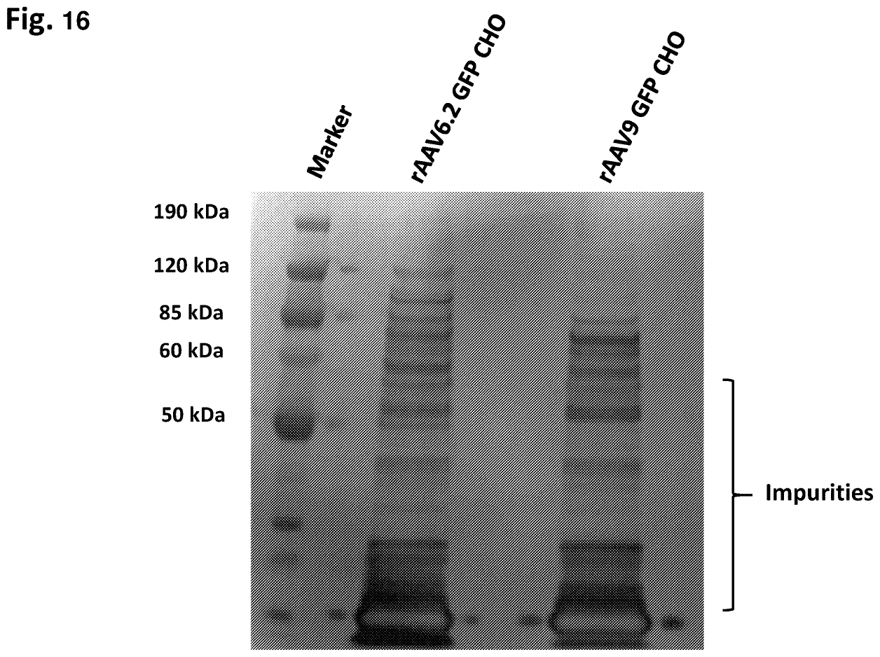

Using the in-house developed PEG-chloroform purification method followed by Amicon concentration produced purified rAAV6.2-GFP and rAAV9-GFP vectors that showed high potency in both in vitro and in vivo studies. The purification method is simple, cheap and fast for production of rAAVs with titers suitable particularly for pre-clinical studies and comparable with the more time-consuming iodixanol ultracentrifugation method and this is in agreement with other studies (Wu et al., 2001. Chin.Sci.Bull. 46, 485-488; Negrini et al., Curr Protoc Neurosci. 2020 September; 93(1):e103). However, impurities were found in the purified samples during CE-SDS testing. These impurities were confirmed by silver staining of the same samples (FIG. 16), which indicates PEG-Chloroform is ideal for preparation of rAAVs samples for in vitro studies, but not for preparing samples for in vivo or clinical studies. Our observation is in agreement with a recent study (Kimura et al., Sci Rep. 2019 Sep. 19; 9(1):13601).

Analysis of the ratio of capsid proteins (VP1:VP2:VP3) of different rAAVs, such as rAAV6.2-GFP and rAAV9-GFP produced in CHO-HV-C1 cells using in-house developed CE-SDS method showed that rAAVs-derived from CHO have very comparable VP1:VP2:VP3 capsid ratio, compared to positive AAV6.2 control that was produced by triple transient transfection system. This indicates rAAVs produced in CHO-HV-C1 cells are fully packaged, compared to other platforms that report the need for genetic engineering to enhance VP expression of some AAV serotypes production in insect cells using baculovirus system. For example, early trials to adapt AAV-5 for production in insect cells using baculovirus system showed low levels of VP1 incorporation into the capsid (Urabe et al., J Virol. 2006; 80:1874-85; Mietzsch et al., Hum Gene Ther. 2014; 25:212-22). Interestingly, examination of full/empty capsids for rAAVs produced in CHO-HV-C1 clone showed higher full capsid percentage than that has been reported before (Small et al., Mol Ther Methods Clin Dev. 2016 May 11; 3:16031). Moreover, testing rAAVs-derived from CHO cells in vitro showed high infectivity titers, compared to those produced by triple transient transfection. In addition, rAAVs-derived from CHO showed very comparable biodistribution in mice, particularly for rAAV9-GFP compared to those produced by triple transient transfection.

To address the second challenge in scaling-up rHSV1 vector stock, CHO-HV-C1 cells were re-engineered to express rHSV-1 Chinese hamster codon-optimized ICP27 protein using both random integration and/or CRISPR/Cas9 technology. Using an in-house medium, the final selected clone (called CHO-HV1-ICP27-C11) showed productive infection for rHSV-1 as indicated replication of viruses propagated on CHO-HV1-ICP27-C11 clone in V27 cells. However, production capacity of rHSV-1 vectors from CHO-HV1-ICP27-C11 was lower, compared to V27 cells. It seems that expression of late HSV-1 viral proteins in the infected CHO-HV1-ICP27 C11 cells was few or below normal levels, compared to expression of early and intermediate viral genes (data not shown). Moreover, it seems likely that CHO cells do not provide the elements necessary for the optimal expression of many HSV genes, particularly late genes because CHO cells may express some inhibitory factors that interfere or block HSV-1 late viral genes expression and our result is in agreement with (Shieh et al., J Cell Biol. 1992 March; 116(5):1273-81). Other factors may be related to the in-house medium used, which can have some inhibitory effect for rHSV-1 vector production. Therefore, in-house serum-free adapted BHK-21 cells were engineered to express HSV-1 ICP27 protein for production of rHSV-1 vectors. Interestingly, stable transfected BHK-21-ICP27 pools produced comparable rHSV-1 titers in Xell medium in presence or absence of FBS.

Thus, the present disclosure provides a rAAV-based HSV production platform in engineered CHO cells that provides a scalable, serum-free manufacturing platform that will facilitate the manufacture of future rAAV-based biotherapeutics in a low-cost manner.

What is claimed is:

1. A baby hamster kidney cell that is adapted to grow in serum-free conditions, wherein the cell stably expresses a hamster codon-optimized herpes simplex virus-1 (HSV-1) ICP27 open reading frame comprising a deletion of non-essential infected-cell protein 27 (ICP27) elements.

2. The cell of claim 1, wherein the cell grows in suspension.

3. The cell of claim 1 or 2, wherein the non-essential ICP27 elements are the 5′ and 3′ untranslated regions (UTRs).

4. A cell line comprising the cell of any one of claims 1-3.

5. A method of producing recombinant adeno-associated virus (rAAV) vectors comprising:
introducing recombinant herpes virus (rHSV) vectors containing AAV rep and cap sequences and sequence encoding a gene of interest into the cell of any one of claims 1-3, or the cell line of claim 4; and culturing the cell or cell line under conditions to produce the rAAV vectors.

6. A Chinese hamster ovary (CHO) cell that is adapted to grow in serum-free conditions, wherein the cell stably expresses one or more polypeptides necessary for entry and infection with herpes simplex virus-1 (HSV-1).

7. The CHO cell of claim 5, wherein the CHO cell stably expresses herpes virus entry mediator (HVEM) and/or Nectin-1.

8. The CHO cell of claim 6, wherein the HVEM and/or nectin-1 sequence has been codon optimized for expression in CHO cells.

9. A cell line comprising the CHO cell of claim 5 or 6.

10. A method of producing recombinant adeno-associated virus (rAAV) vectors comprising:
introducing recombinant herpes virus (rHSV) vectors containing AAV rep and cap sequences and sequence encoding a gene of interest (GOI) into the cell of any one of claims 5-7, or the cell line of claim 8; and culturing the cell or cell line under conditions to produce the rAAV vectors.

11. The method of claim 5 or 9, wherein the rHSV vectors are introduced at a multiplicity of infection of about 4:1, 6:1, 8:1, or 10:1 rHSV-rep cap:rHSV-GOI.

12. The method of any one of claims 5, 9, or 10, wherein the AAV serotype is AAV6, AAV8 or AAV9.

13. The method of claim 5 or 9, wherein the gene of interest encodes any therapeutic biologic compound.

14. The method of claim 12, wherein the biologic compound is an antibody or a chimeric antigen receptor.
